(19) United States
(12) Patent Application Publication
(10) Pub. No.: US 2020/0246393 A1
(43) Pub. Date: Aug. 6, 2020

(54) TUMOR SUPPRESSION USING HUMAN PLACENTA-DERIVED INTERMEDIATE NATURAL KILLER (PINK) CELLS IN COMBINATION WITH AN ANTIBODY

(71) Applicant: CELULARITY, INC., Warren, NJ (US)

(72) Inventors: Xiaokui ZHANG, Livingston, NJ (US); Lin KANG, Edison, NJ (US); Robert J. HARIRI, Bernardsville, NJ (US)

(73) Assignee: CELULARITY, INC., Warren, NJ (US)

(21) Appl. No.: 16/649,133

(22) PCT Filed: Sep. 27, 2018
(86) PCT No.: PCT/US2018/053233

§ 371 Date: Mar. 19, 2020

Related U.S. Application Data
(60) Provisional application No. 62/564,735, filed on Sep. 28, 2017.

(51) Int. Cl. A61K 35/50; A61K 35/17; C07K 16/28; C07K 16/30; C07K 16/32; C12N 5/0783; C12N 5/071; A61K 31/454; A61P 35/00

(52) U.S. Cl. CPC A61K 35/50; A61K 35/17; C07K 16/2887; C07K 16/2896; C07K 16/3084; A61P 35/00; C07K 16/32; C07K 16/2863; C12N 5/0646; C12N 5/0697; A61K 31/454; C07K 16/2827

(57) ABSTRACT

Provided herein are methods of suppressing the growth or proliferation of tumor cells and methods of treating individuals having tumor cells using placental perfusate, placental perfusate cells, placenta-derived intermediate natural killer (PINK) cells, combined natural killer (NK) cells, or combinations thereof, in combination with an antibody (e.g., anti-disialoganglioside (anti-GD2) antibody). Also provided herein are compositions comprising placental perfusate, placental perfusate cells, PINK cells, combined NK cells, or combinations thereof, in combination with an antibody (e.g., anti-GD2 antibody).

This application claims priority to U.S. Provisional Patent Application No. 62/564,735, filed Sep. 28, 2017, the disclosure of which is incorporated herein by reference in its entirety.

### 1. FIELD

Presented herein are methods of suppressing the growth or proliferation of tumor cells by contacting the tumor cells with human placental perfusate, placental perfusate cells, natural killer (NK) cells derived from placenta (e.g., NK cells isolated from placental perfusate or NK cells differentiated from CD34⁺ hematopoietic stem cells recovered from placental perfusate), and/or combined NK cells comprising NK cells derived from placenta (e.g., NK cells isolated from placental perfusate or NK cells differentiated from CD34⁺ hematopoietic stem cells recovered from placental perfusate) and NK cells from umbilical cord blood, in combination with an antibody (e.g., anti-disialoganglioside (anti-GD2) antibody). Also provided herein are methods of treating individuals having tumor cells by administering to the individuals a therapeutically effective amount of human placental perfusate, placental perfusate cells, NK cells derived from placenta (e.g., NK cells isolated from placental perfusate or NK cells differentiated from CD34⁺ hematopoietic stem cells recovered from placental perfusate), and/or combined NK cells comprising NK cells derived from placenta (e.g., NK cells isolated from placental perfusate or NK cells differentiated from CD34⁺ hematopoietic stem cells recovered from placental perfusate) and NK cells from umbilical cord blood, in combination with an antibody (e.g., anti-GD2 antibody). Further provided herein are compositions comprising human placental perfusate, placental perfusate cells, NK cells derived from placenta (e.g., NK cells isolated from placental perfusate or NK cells differentiated from CD34⁺ hematopoietic stem cells recovered from placental perfusate), and/or combined NK cells comprising NK cells derived from placenta (e.g., NK cells isolated from placental perfusate or NK cells differentiated from CD34⁺ hematopoietic stem cells recovered from placental perfusate) and NK cells from umbilical cord blood, in combination with an antibody (e.g., anti-GD2 antibody).

### 2. BACKGROUND

Placental perfusate comprises a collection of placental cells obtained by passage of a perfusion solution through the placental vasculature, and collection of the perfusion fluid from the vasculature, from the maternal surface of the placenta, or both. Methods of perfusing mammalian placentas are described, e.g., in U.S. Pat. Nos. 7,045,146 and 7,255,879. The population of placental cells obtained by perfusion is heterogenous, comprising hematopoietic (CD34⁺) cells, nucleated cells such as granulocytes, monocytes and macrophages, a small percentage (less than 1%) tissue culture substrate-adherent placental stem cells, and natural killer cells.

Natural killer (NK) cells are cytotoxic lymphocytes that constitute a major component of the innate immune system. NK cells do not express T-cell antigen receptors (TCR), CD3 or surface immunoglobulins (Ig) B cell receptor, but usually express the surface markers CD16 (FcγRIII) and CD56 in humans. NK cells are cytotoxic; small granules in their cytoplasm contain special proteins such as perforin and proteases known as granzymes. Upon release in close proximity to a cell slated for killing, perforin forms pores in the cell membrane of the target cell through which the granzymes and associated molecules can enter, inducing apoptosis. One granzyme, granzyme B (also known as granzyme 2 and cytotoxic T-lymphocyte-associated serine esterase 1), is a serine protease crucial for rapid induction of target cell apoptosis in the cell-mediated immune response.

NK cells are activated in response to interferons or macrophage-derived cytokines. Activated NK cells are referred to as lymphokine activated killer (LAK) cells. NK cells possess two types of surface receptors, labeled “activating receptors” and “inhibitory receptors,” that control the cells' cytotoxic activity.

Among other activities, NK cells play a role in the host rejection of tumors. Because cancer cells have reduced or no class I MHC expression, they can become targets of NK cells. Accumulating clinical data suggest that haploidentical transplantation of human NK cells isolated from PBMC or bone marrow mediate potent anti-leukemia effects without possessing detectable graft versus host disease (GVHD). See Ruggeri et al., Science 295:2097-2100 (2002)). Natural killer cells can become activated by cells lacking, or displaying reduced levels of, major histocompatibility complex (MHC) proteins. Activated and expanded NK cells and LAK cells have been used in both ex vivo therapy and in vivo treatment of patients having advanced cancer, with some success against bone marrow related diseases, such as leukemia; breast cancer; and certain types of lymphoma.

### 3. SUMMARY

Provided herein are methods of using human placental perfusate, placental perfusate cells, NK cells derived from placenta (e.g., NK cells isolated from placental perfusate or NK cells differentiated from CD34⁺ hematopoietic stem cells recovered from placental perfusate), and/or combined NK cells comprising NK cells derived from placenta (e.g., NK cells isolated from placental perfusate or NK cells differentiated from CD34⁺ hematopoietic stem cells recovered from placental perfusate) and NK cells from umbilical cord blood, in combination with an antibody (e.g., anti-GD2 antibody) in suppressing tumor cell proliferation or treating individuals having tumor cells. Also provided herein are compositions comprising human placental perfusate, placental perfusate cells, NK cells derived from placenta (e.g., NK cells isolated from placental perfusate or NK cells differentiated from CD34⁺ hematopoietic stem cells recovered from placental perfusate), and/or combined NK cells comprising NK cells derived from placenta (e.g., NK cells isolated from placental perfusate or NK cells differentiated from CD34⁺ hematopoietic stem cells recovered from placental perfusate) and NK cells from umbilical cord blood, in combination with an antibody (e.g., anti-GD2 antibody).

In one aspect, provided herein is a method of suppressing the proliferation of tumor cells, comprising contacting the tumor cells with human placental perfusate cells and an antibody, wherein said placental perfusate cells comprise CD56⁺ NK cells.

In certain embodiments, the tumor cells are blood cancer cells.

In some embodiments, the tumor cells are solid tumor cells.

In other embodiments, the tumor cells are glioblastoma cells, neuroblastoma cells, osteosarcoma cells, melanoma cells, ovarian cancer cells, primary ductal carcinoma cells, leukemia cells, acute T cell leukemia cells, acute myelogenous leukemia (AML) cells, chronic myelogenous leukemia (CML) cells, acute lymphocytic leukemia (ALL) cells, chronic lymphocytic leukemia (CLL) cells, non-hodgkin's lymphoma (NHL) cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

In certain embodiments, said contacting is contacting in vitro.

In some embodiments, said contacting is contacting in vivo.

In other embodiments, said contacting is in a human.

In one embodiment, said placental perfusate cells are total nucleated cells from placental perfusate.

In another embodiment, said placental perfusate cells comprise at least about 50% CD56⁺ NK cells.

In another aspect, provided herein is a method of suppressing the proliferation of tumor cells comprising contacting the tumor cells with human placental perfusate cells and an antibody, wherein said placental perfusate cells comprise CD56⁺, CD16⁻ PINK cells.

In certain embodiments, said contacting takes place in vitro.

In some embodiments, said contacting takes place in vivo.

In other embodiments, said contacting takes place in a human.

In yet other embodiments, said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

In still other embodiments, said PINK cells are contacted with an immunomodulatory compound in an amount and for a time sufficient for said PINK cells to express detectably more granzyme B than an equivalent number of PINK cells not contacted with said immunomodulatory compound.

In one embodiment, said immunomodulatory compound is lenalidomide, pomalidomide, or thalidomide. In one embodiment, said immunomodulatory compound is lenalidomide. In another embodiment, said immunomodulatory compound is pomalidomide. In yet another embodiment, said immunomodulatory compound is thalidomide.

In yet another aspect, provided herein is a method of suppressing the proliferation of tumor cells comprising contacting the tumor cells with combined NK cells and an antibody, wherein said combined NK cells comprise NK cells isolated from placental perfusate and NK cells isolated from umbilical cord blood, and wherein said umbilical cord blood is isolated from the placenta from which said placental perfusate is obtained.

In certain embodiments, said contacting takes place in vitro.

In some embodiments, said contacting takes place in vivo.

In other embodiments, said contacting takes place in a human.

In yet other embodiments, said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

In certain embodiments, said combined NK cells comprise:

a detectably higher number of CD3⁻CD56⁺CD16⁻ NK cells than an equivalent number of NK cells from peripheral blood;
a detectably lower number of CD3⁻CD56⁺CD16⁺ NK cells than an equivalent number of NK cells from peripheral blood;
a detectably higher number of CD3⁻CD56⁺KIR2DL2/L3⁺ NK cells than an equivalent number of NK cells from peripheral blood;
a detectably lower number of CD3⁻CD56⁻NKp46⁺ NK cells than an equivalent number of NK cells from peripheral blood;
a detectably higher number of CD3⁻CD56⁺NKp30⁺ NK cells than an equivalent number of NK cells from peripheral blood;
a detectably higher number of CD3⁻CD56⁺2B4⁺ NK cells than an equivalent number of NK cells from peripheral blood; or
a detectably higher number of CD3⁻CD56⁺CD94⁺ NK cells than an equivalent number of NK cells from peripheral blood.

In one embodiment, said NK cells have not been cultured.

In other embodiments, said combined NK cells comprise:

a detectably lower number of CD3⁻CD56⁺KIR2DL2/L3⁺ NK cells than an equivalent number of NK cells from peripheral blood;
a detectably higher number of CD3⁻CD56⁺NKp46⁺ NK cells than an equivalent number of NK cells from peripheral blood;
a detectably higher number of CD3⁻CD56⁺NKp44⁺ NK cells than an equivalent number of NK cells from peripheral blood;
a detectably higher number of CD3⁻CD56⁺NKp30⁺ NK cells than an equivalent number of NK cells from peripheral blood.

In one embodiment, said NK cells have been cultured.

In another embodiment, said NK cells have been cultured for about 21 days.

In still another aspect, provided herein is a method of treating an individual having tumor cells, comprising administering to said individual a therapeutically effective amount of human placental perfusate cells and an antibody, wherein said human placental perfusate cells comprise CD56⁺ NK cells.

In certain embodiments, the tumor cells are blood cancer cells.

In some embodiments, the tumor cells are solid tumor cells.

In other embodiments, the tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

In certain embodiments, said human placental perfusate cells and/or said antibody are administered intravenously. In one embodiment, said human placental perfusate cells are administered intravenously. In another embodiment, said antibody is administered intravenously. In yet another embodiment, said human placental perfusate cells and said antibody are administered intravenously.

In some embodiments, said human placental perfusate cells and/or said antibody are administered intracranially. In one embodiment, said human placental perfusate cells are administered intracranially. In another embodiment, said antibody is administered intracranially. In yet another embodiment, said human placental perfusate cells and said antibody are administered intracranially.

In other embodiments, said human placental perfusate cells and/or said antibody are administered intrathecally. In one embodiment, said human placental perfusate cells are administered intrathecally. In another embodiment, said antibody is administered intrathecally. In yet another embodiment, said human placental perfusate cells and said antibody are administered intrathecally.

In certain embodiments, said human placental perfusate cells and said antibody are administered concurrently. In some embodiments, said human placental perfusate cells and said antibody are administered sequentially. In one embodiment, said human placental perfusate cells are administered before said antibody. In another embodiment, said human placental perfusate cells are administered after said antibody.

In some embodiments, said human placental perfusate cells are total nucleated cells from placental perfusate.

In other embodiments, said human placental perfusate cells comprise at least about 50% CD56⁺ NK cells.

In yet other embodiments, said NK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, or hsa-miR-99a at a detectably higher level than peripheral blood NK cells.

In still other embodiments, said NK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

In another aspect, provided herein a method of treating an individual having tumor cells comprising administering to said individual a therapeutically effective amount of human placental perfusate cells and an antibody, wherein said human placental perfusate cells comprise CD56⁺, CD16⁻ PINK cells.

In certain embodiments, said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

In some embodiments, said PINK cells are contacted with an immunomodulatory compound in an amount and for a time sufficient for said PINK cells to express detectably more granzyme B than an equivalent number of PINK cells not contacted with said immunomodulatory compound.

In certain embodiments, said immunomodulatory compound is lenalidomide, pomalidomide, or thalidomide. In one embodiment, said immunomodulatory compound is lenalidomide. In another embodiment, said immunomodulatory compound is pomalidomide. In yet another embodiment, said immunomodulatory compound is thalidomide.

In certain embodiments, said human placental perfusate cells and/or said antibody are administered intravenously. In one embodiment, said human placental perfusate cells are administered intravenously. In another embodiment, said antibody is administered intravenously. In yet another embodiment, said human placental perfusate cells and said antibody are administered intravenously.

In some embodiments, said human placental perfusate cells and/or said antibody are administered intracranially. In one embodiment, said human placental perfusate cells are administered intracranially. In another embodiment, said antibody is administered intracranially. In yet another embodiment, said human placental perfusate cells and said antibody are administered intracranially.

In other embodiments, said human placental perfusate cells and/or said antibody are administered intrathecally. In one embodiment, said human placental perfusate cells are administered intrathecally. In another embodiment, said antibody is administered intrathecally. In yet another embodiment, said human placental perfusate cells and said antibody are administered intrathecally.

In certain embodiments, said human placental perfusate cells and said antibody are administered concurrently. In some embodiments, said human placental perfusate cells and said antibody are administered sequentially. In one embodiment, said human placental perfusate cells are administered before said antibody. In another embodiment, said human placental perfusate cells are administered after said antibody.

In some embodiments, said human placental perfusate cells are total nucleated cells from placental perfusate.

In certain embodiments, said human placental perfusate cells comprise at least about 50% CD56⁺, CD16⁻ PINK cells.

In some embodiments, said PINK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, or hsa-miR-99a at a detectably higher level than peripheral blood NK cells.

In other embodiments, said PINK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

In yet another aspect, provided herein is a method of treating an individual having tumor cells, comprising administering to said individual a therapeutically effective amount of combined NK cells and an antibody, wherein said combined NK cells comprise NK cells isolated from placental perfusate and NK cells isolated from umbilical cord blood, and wherein said umbilical cord blood is isolated from the placenta from which said placental perfusate is obtained.

In certain embodiments, said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

In some embodiments, said combined NK cells comprise:

a detectably higher number of CD3−CD56+CD16−NK cells than an equivalent number of NK cells from peripheral blood;
a detectably lower number of CD3−CD56+CD16+NK cells than an equivalent number of NK cells from peripheral blood;
a detectably higher number of CD3−CD56+KIR2DL2/L3+NK cells than an equivalent number of NK cells from peripheral blood;
a detectably lower number of CD3−CD56+NKp46+NK cells than an equivalent number of NK cells from peripheral blood;
a detectably higher number of CD3−CD56+NKp30+NK cells than an equivalent number of NK cells from peripheral blood;
a detectably higher number of CD3−CD56+2B4+NK cells than an equivalent number of NK cells from peripheral blood; or
a detectably higher number of CD3−CD56+CD94+NK cells than an equivalent number of NK cells from peripheral blood.

In one embodiment, said NK cells have not been cultured.

In other embodiments, said combined NK cells comprise:

a detectably lower number of CD3⁻CD56⁺KIR2DL2/L3⁺ NK cells than an equivalent number of NK cells from peripheral blood;
a detectably higher number of CD3⁻CD56⁺NKp46⁺ NK cells than an equivalent number of NK cells from peripheral blood;
a detectably higher number of CD3⁻CD56⁺NKp44⁺ NK cells than an equivalent number of NK cells from peripheral blood;
a detectably higher number of CD3⁻CD56⁺NKp30⁺ NK cells than an equivalent number of NK cells from peripheral blood.

In one embodiment, said NK cells have been cultured.

In another embodiment, said NK cells have been cultured for about 21 days.

In certain embodiments, said combined NK cells and/or said antibody are administered intravenously. In one embodiment, said combined NK cells are administered intravenously. In another embodiment, said antibody is administered intravenously. In yet another embodiment, said combined NK cells and said antibody are administered intravenously.

In some embodiments, said combined NK cells and/or said antibody are administered intracranially. In one embodiment, said combined NK cells are administered intracranially. In another embodiment, said antibody is administered intracranially. In yet another embodiment, said combined NK cells and said antibody are administered intracranially.

In other embodiments, said combined NK cells and/or said antibody are administered intrathecally. In one embodiment, said combined NK cells are administered intrathecally. In another embodiment, said antibody is administered intrathecally. In yet another embodiment, said combined NK cells and said antibody are administered intrathecally.

In certain embodiments, said combined NK cells and said antibody are administered concurrently. In some embodiments, said combined NK cells and said antibody are administered sequentially. In one embodiment, said combined NK cells are administered before said antibody. In another embodiment, said combined NK cells are administered after said antibody.

In certain embodiments, said NK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, or hsa-miR-99a at a detectably higher level than peripheral blood NK cells.

In other embodiments, said NK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

In one aspect, provided herein is a method of treating an individual comprising tumor cells comprising administering to said individual a therapeutically effective amount of CD56⁺, CD16⁻ PINK cells and an antibody.

In certain embodiments, the CD56⁺, CD16⁻ PINK cells are generated from CD34⁺ hematopoietic stem cells by a two-step expansion and differentiation method.

In some embodiments, said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

In other embodiments, said PINK cells are contacted with an immunomodulatory compound in an amount and for a time sufficient for said PINK cells to express detectably more granzyme B than an equivalent number of PINK cells not contacted with said immunomodulatory compound.

In certain embodiments, said immunomodulatory compound is lenalidomide, pomalidomide, or thalidomide. In one embodiment, said immunomodulatory compound is lenalidomide. In another embodiment, said immunomodulatory compound is pomalidomide. In yet another embodiment, said immunomodulatory compound is thalidomide.

In certain embodiments, said PINK cells and/or said antibody are administered intravenously. In one embodiment, said PINK cells are administered intravenously. In another embodiment, said antibody is administered intravenously. In yet another embodiment, said PINK cells and said antibody are administered intravenously.

In some embodiments, said PINK cells and/or said antibody are administered intracranially. In one embodiment, said PINK cells are administered intracranially. In another embodiment, said antibody is administered intracranially. In yet another embodiment, said PINK cells and said antibody are administered intracranially.

In other embodiments, said PINK cells and/or said antibody are administered intrathecally. In one embodiment, said PINK cells are administered intrathecally. In another embodiment, said antibody is administered intrathecally. In yet another embodiment, said PINK cells and said antibody are administered intrathecally.

In certain embodiments, said PINK cells and said antibody are administered concurrently. In some embodiments, said PINK cells and said antibody are administered sequentially. In one embodiment, said PINK cells are administered before said antibody. In another embodiment, said PINK cells are administered after said antibody.

In certain embodiments of various methods provided herein, said antibody is selected from the group consisting of an anti-disialoganglioside (anti-GD2) antibody, an anti-CD38 antibody, an anti-SLAMF7 antibody, an anti-CD 20 antibody, an anti-HER2 antibody, an anti-PD-L1 antibody, and an anti-EGFR antibody.

In some embodiments, the antibody is an anti-GD2 antibody.

In a specific embodiment, the anti-GD2 antibody is dinutuximab.

In other embodiments, the antibody is an anti-CD38 antibody.

In a specific embodiment, the anti-CD38 antibody is daratumumab.

In yet other embodiments, the antibody is an anti-SLAMF7 antibody.

In a specific embodiment, the anti-SLAMF7 antibody is elotuzumab.

In still other embodiments, the antibody is an anti-CD 20 antibody.

In a specific embodiment, the anti-CD 20 antibody is obinutuzumab, ofatumumab, or rituximab. In one embodiment, the anti-CD20 antibody is obinutuzumab. In another embodiment, the anti-CD20 antibody is ofatumumab. In yet another embodiment, the anti-CD20 antibody is rituximab.

In certain embodiments, the antibody is an anti-HER2 antibody.

In a specific embodiment, the anti-HER2 antibody is trastuzumab.

In some embodiments, the antibody is an anti-PD-L1 antibody.

In a specific embodiment, the anti-PD-L1 antibody is atezolizumab or avelumab. In one embodiment, the anti-PD-L1 antibody is atezolizumab. In another embodiment, the anti-PD-L1 antibody is avelumab.

In other embodiments, the antibody is an anti-EGFR antibody.

In a specific embodiment, the anti-EGFR antibody is cetuximab.

### 4. BRIEF DESCRIPTION OF THE FIGURES

FIG. 1 depicts expression of GD2 on various solid tumor cell lines.

FIGS. 2A and 2B show that the cytotoxicity of PNK cells against neuroblastoma cell line NMB-7 increased significantly in presence of Unituxin®, compared with that in presence of IgG1 control, at E:T ratio of 10:1 (A) or 3:1 (B).

FIGS. 3A-3C show that the cytotoxicity of PNK cells against human lung cancer cell line SK-MES-1 (A), human melanoma cell line HT-144 (B), or human ovarian cancer cell line Coav-3 (C), increased significantly in presence of Unituxin®, compared with that in presence of IgG1 control, at E:T ratio of 3:1.

FIGS. 4A and 4B show that, in presence of Unituxin®, the cytotoxicity of PNK cells against human glioblastoma cell lines U-251 (A) increased significantly, and the cytotoxicity of PNK cells against human glioblastoma cell lines U-87MG (B) likewise increased cytotoxicity, compared with that in presence of IgG1 control, at E:T ratio of 1:1.

FIG. 5 shows that human lymphoma cell lines, Daudi, HS-sultan, and Raji, express CD38.

FIGS. 6A-6C show that, in presence of daratumumab, the cytotoxicity of PNK cells against Daudi cell line (A) and HS-Sultan cell line (B) significantly increased but the cytotoxicity of PNK cells against Raji cell line (C) did not change much, compared with that in presence of IgG1 control, at E:T ratio of 1:1.

FIG. 7 shows increased IFN-γ secretion of PNK cells in presence of daratumumab-pretreated HS-sultan cells, compared with that of PNK cells alone or PNK cells in presence of IgG1-pretreated HS-sultan cells.

FIG. 8 shows CD38 expression on PNK cells from three donors.

FIG. 9 shows that daratumumab had no significant effect on viability of PNK cells from three donors, in comparison with human IgG control.

FIG. 10 shows CD20 expression on human lymphoma Daudi cell line.

FIG. 11 shows that the cytotoxicity of PNK cells against lymphoma cell line Daudi increased significantly in presence of Rituxan®, compared with that in presence of IgG1 control, at E:T ratio of 1:1.

FIG. 12 depicts tumor growth kinetics in orthotopic glioblastoma model mice injected with different numbers of U87 MG-Red-FLuc cells.

### 5. DETAILED DESCRIPTION

Provided herein are methods of suppressing growth or proliferation of tumor cells and methods of treating individuals having tumor cells using placental perfusate, placental perfusate cells, PINK cells, and/or combined NK cells, in combination with an antibody (e.g., an anti-GD2 antibody). Also provided herein are compositions comprising placental perfusate, placental perfusate cells, PINK cells, and/or combined NK cells, in combination with an antibody (e.g., an anti-GD2 antibody), and methods of using such compositions.

### 5.1. Definitions

As used herein, “combined NK cells” are NK cells, e.g., from matched umbilical cord and human placental perfusate, wherein placental perfusate is obtained from the same placenta as the cord blood. NK cells from both are isolated separately or at the same time, and combined.

As used herein, “PINK,” “PINK cells,” “placental intermediate NK cells,” “PNK cells,” or “placenta-derived intermediate NK cells” refers to NK cells that are obtained from human placenta, e.g., human placental perfusate or placental tissue that has been mechanically and/or enzymatically disrupted, or generated by a two-step expansion and differentiation method using hematopoietic stem cells that are recovered from human placenta, as exemplified in U.S. Pat. No. 8,926,964. PINK cells are CD56⁺ and CD16⁻, e.g., as determined by flow cytometry, e.g., fluorescence-activated cell sorting using antibodies to CD56 and CD16. PINK cells are not obtained from umbilical cord blood or peripheral blood. In some specific embodiments, examples of PNK cells include PNK-007 cells.

As used herein, “placental perfusate” means perfusion solution that has been passed through at least part of a placenta, e.g., a human placenta, e.g., through the placental vasculature, including a plurality of cells collected by the perfusion solution during passage through the placenta.

As used herein, “placental perfusate cells” means nucleated cells, e.g., total nucleated cells, isolated from, or isolatable from, placental perfusate.

As used herein, “tumor cell suppression,” “suppression of tumor cell proliferation,” “suppressing growth of tumor cells,” and the like, refer to slowing the growth of a population of tumor cells, e.g., by killing one or more of the tumor cells in said population of tumor cells, for example, by contacting the population of tumor cells with PINK cells, a population of cells comprising PINK cells, combined NK cells, a population of cells comprising combined NK cells, human placental perfusate, or the like.

### 5.2. Placental Perfusate and Placental Perfusate Cells

Placental perfusate comprises a heterogeneous collection of cells. Typically, placental perfusate is depleted of erythrocytes prior to use. Such depletion can be carried out by known methods of separating red blood cells from nucleated blood cells. In certain embodiment, the perfusate or perfusate cells are cryopreserved. In certain other embodiments, the placental perfusate comprises, or the placental perfusate cells comprise, only fetal cells, or a combination of fetal cells and maternal cells.

Typically, placental perfusate from a single placental perfusion comprises about 100 million to about 500 million nucleated cells. In certain embodiments, the placental perfusate or perfusate cells comprise CD34⁺ cells, e.g., hematopoietic stem or progenitor cells. Such cells can, in a more specific embodiment, comprise CD34⁺CD45⁻ stem or progenitor cells, CD34⁺CD45⁺ stem or progenitor cells, myeloid progenitors, lymphoid progenitors, and/or erythroid progenitors. In other embodiments, placental perfusate and placental perfusate cells comprise adherent placental stem cells, e.g., CD34⁻ stem cells. In other embodiment, the placental perfusate and placental perfusate cells comprise, e.g., endothelial progenitor cells, osteoprogenitor cells, and natural killer cells. In certain embodiments, placental perfusate as collected from the placenta and depleted of erythrocytes, or perfusate cells isolated from such perfusate, comprise about 6-7% natural killer cells (CD3⁻, CD56⁺); about 21-22% T cells (CD3⁺); about 6-7% B cells (CD19⁺); about 1-2% endothelial progenitor cells (CD34⁺, CD31⁺); about 2-3% neural progenitor cells (nestin⁺); about 2-5% hematopoietic progenitor cells (CD34⁺); and about 0.5-1.5% adherent placental stem cells (e.g., CD34⁻, CD117⁻, CD105⁺ and CD44⁺), as determined, e.g. by flow cytometry, e.g., by FACS analysis.

### 5.3. Placental NK Cells

The isolation, characterization, and use of NK cells obtainable from placenta, e.g., from placental perfusate and/or from mechanically and/or enzymatically-disrupted placental tissue, are disclosed in U.S. Pat. No. 8,263,065, which is incorporated by reference herein in its entirety. Placental NK cells can also be generated by a two-step expansion and differentiation method using hematopoietic stem cells, as disclosed in U.S. Pat. No. 8,926,964, which is incorporated by reference herein in its entirety.

In a specific embodiment, the placental NK cells are “placental intermediate NK cells” or “PINK cells,” which are characterized as being CD56⁺CD16⁻, i.e., displaying the CD56 cellular marker and lacking the CD16 cellular marker, e.g., as determined by flow cytometry, e.g., fluorescence-activated cell sorting using antibodies against CD16 and CD56, as described above. In certain embodiments, the PINK cells are isolated from placenta. In some embodiments, the PINK cells are isolated from placental perfusate. In some embodiments, the PINK cells are isolated from placental perfusate cells. In some embodiments, the PINK cells are generated by a two-step expansion and differentiation method using hematopoietic stem cells. In certain embodiments, the hematopoietic stem cells are CD34⁺. In other embodiments, the hematopoietic stem cells are isolated from placenta. In yet other embodiments, the hematopoietic stem cells are isolated from placental perfusate. In some embodiments, the PINK cells are generated by a two-step expansion and differentiation method using CD34⁺ hematopoietic stem cells recovered from placenta. In some embodiments, the PINK cells are generated by a two-step expansion and differentiation method using CD34⁺ hematopoietic stem cells recovered from placental perfusate. In some embodiments, a plurality of NK cells comprises CD56⁺CD16⁻ PINK cells in combination with CD56⁺CD16⁺ NK cells. In more specific embodiments, the CD56⁺CD16⁺ NK cells can be isolated from placenta, or from another source, e.g., peripheral blood, umbilical cord blood, bone marrow, or the like. Thus, in various other embodiments, PINK cells can be combined with CD56⁺CD16⁺ NK cells, e.g., in ratios of, for example, about 1:10, 2:9, 3:8, 4:7, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or about 9:1. As used in this context, “isolated” means that the cells have been removed from their normal environment, e.g., the placenta.

In certain embodiments, the PINK cells are CD3⁻.

In other embodiments, the PINK cells do not exhibit one or more cellular markers exhibited by fully mature NK cells (e.g., CD16), or exhibit such one or more markers at a detectably reduced level compared to fully mature NK cells, or exhibit one or more cellular markers associated with NK cell precursors but not fully mature NK cells. In a specific embodiment, a PINK cell provided herein expresses NKG2D, CD94 and/or NKp46 at a detectably lower level than a fully mature NK cell. In another specific embodiment, a plurality of PINK cells provided herein expresses, in total, NKG2D, CD94 and/or NKp46 at a detectably lower level than an equivalent number of fully mature NK cells.

In certain embodiments, PINK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-5¹⁵-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, and/or hsa-miR-99a at a detectably higher level than peripheral blood NK cells.

In some embodiments, said PINK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

In certain embodiments, the placental NK cells, e.g., PINK cells, have been expanded in culture. In certain other embodiments, the placental perfusate cells have been expanded in culture. In a specific embodiment, said placental perfusate cells have been expanded in the presence of a feeder layer and/or in the presence of at least one cytokine. In a more specific embodiment, said feeder layer comprises K562 cells or peripheral blood mononuclear cells. In another more specific embodiment, said at least one cytokine is interleukin-2.

In another embodiment, provided herein is an isolated plurality (e.g., population) of PINK cells. In another specific embodiment, the isolated population of cells is produced by CD56-microbead isolation of cells from placental perfusate. In various specific embodiments, the population comprises at least about 50%, 55%, 60%, 65%, 70%, 75%, 80%, 85%, 90%, 95%, 98% or at least about 99% PINK cells. In another embodiment, the plurality of PINK cells comprises, or consists of, PINK cells that have not been expanded; e.g., are as collected from placental perfusate. In another embodiment, the plurality of PINK cells comprises, or consists of, PINK cells that have been expanded. Methods of expanding NK cells have been described, e.g., in Ohno et al., U.S. Patent Application Publication No. 2003/0157713; see also Yssel et al., J. Immunol. Methods 72(1):219-227 (1984) and Litwin et al., J. Exp. Med. 178(4):1321-1326 (1993) and the description of NK cell expansion in Example 1, below.

In other embodiments, the isolated plurality of PINK cells does not exhibit one or more cellular markers exhibited by fully mature NK cells (e.g., CD16), or exhibits such one or more markers at a detectably reduced level compared to fully mature NK cells, or exhibits one or more cellular markers associated with NK cell precursors but not associated with fully mature NK cells. In a specific embodiment, a PINK cell provided herein expresses NKG2D, CD94 and/or NKp46 at a detectably lower level than a fully mature NK cell. In another specific embodiment, a plurality of PINK cells provided herein expresses, in total, NKG2D, CD94 and/or NKp46 at a detectably lower level than an equivalent number of fully mature NK cells.

In certain specific embodiments, the population of PINK cells expresses one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-5¹⁵-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, and/or hsa-miR-99a at a detectably higher level than peripheral blood NK cells. In another specific embodiment, the population of PINK cells expresses a detectably higher amount of granzyme B than an equivalent number of peripheral blood NK cells.

In other embodiments, the PINK cells provided herein have been expanded in culture. In specific embodiments, the PINK cells have been cultured, e.g., expanded in culture, for at least, about, or at most 1, 2, 3, 4, 5, 6, 7, 8, 9, 10, 11, 12, 13, 14, 15, 16, 17, 18, 19, 20, 21, 22, 23, 24, 25, 26, 27 or 28 days. In a specific embodiment, the PINK cells are cultured for about 21 days.

In another embodiment, provided herein is an isolated population of cells, e.g., placental cells, comprising PINK cells. In a specific embodiment, the isolated population of cells is total nucleated cells from placental perfusate, e.g., placental perfusate cells, comprising autologous, isolated PINK cells. In another specific embodiment, the population of cells is an isolated population of cells produced by CD56-microbead isolation of cells from placental perfusate. In various specific embodiments, the population comprises at least about 50%, 55%, 60%, 65%, 70%, 75%, 80%, 85%, 90%, 95%, 98% or at least about 99% PINK cells.

Because the post-partum placenta comprises tissue and cells from the fetus and from the mother placental perfusate, depending upon the method of collection, can comprise fetal cells only, or a substantial majority of fetal cells (e.g., greater than about 90%, 95%, 98% or 99%), or can comprise a mixture of fetal and maternal cells (e.g., the fetal cells comprise less than about 90%, 80%, 70%, 60%, or 50% of the total nucleated cells of the perfusate). In one embodiment, the PINK cells are derived only from fetal placental cells, e.g., cells obtained from closed-circuit perfusion of the placenta (see above) wherein the perfusion produces perfusate comprising a substantial majority, or only, fetal placental cells. In another embodiment, the PINK cells are derived from fetal and maternal cells, e.g., cells obtained by perfusion by the pan method (see above), wherein the perfusion produced perfusate comprising a mix of fetal and maternal placental cells. Thus, in one embodiment, provided herein is a population of placenta-derived intermediate NK cells, the substantial majority of which have the fetal genotype. In another embodiment, provided herein is a population of placenta-derived intermediate NK cells that comprise NK cells having the fetal genotype and NK cells having the maternal phenotype.

Also provided herein are populations of placenta-derived intermediate NK cells that comprise NK cells from a non-placental source. For example, in one embodiment, provided herein is population of PINK cells that also comprises NK cells from umbilical cord blood, peripheral blood, bone marrow, or a combination of two or more of the foregoing. The populations of NK cells comprising PINK cells and NK cells from a non-placental source can comprise the cells in, e.g., a ratio of about 1:10, 2:9, 3:8, 4:7, 5:6, 6:5, 7:4, 8:3, 9:2, 10:1, 1:9, 1:8, 1:7, 1:6, 1:5, 1:4, 1:3, 1:2, 1:1, 2:1, 3:1, 4:1, 5:1, 6:1, 7:1, 8:1, 9:1, 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, or about 1:100, or the like.

Further provided herein are combinations of umbilical cord blood and isolated PINK cells. In various embodiments, cord blood is combined with PINK cells at about 1×10⁴, 5×10⁴, 1×10⁵, 5×10⁵, 1×10⁶, 5×10⁶, 1×10⁷, 5×10⁷, 1×10⁸, or 5×10⁸, or more, PINK cells per milliliter of cord blood.

In one embodiment, PINK cells are collected by obtaining placental perfusate, then contacting the placental perfusate with a composition that specifically binds to CD56⁺ cells, e.g., an antibody against CD56, followed by isolating of CD56⁺ cells on the basis of said binding to form a population of CD56⁺ cells. The population of CD56⁺ cells comprises an isolated population of NK cells. In a specific embodiment, CD56⁺ cells are contacted with a composition that specifically binds to CD16⁺ cells, e.g., an antibody against CD16, and the CD16⁺ cells from the population of CD56⁺ cells. In another specific embodiment, CD3⁺ cells are also excluded from the population of CD56⁺ cells.

In one embodiment, PINK cells are obtained from placental perfusate as follows. Post-partum human placenta is exsanguinated and perfused, e.g., with about 200-800 mL of perfusion solution, through the placental vasculature only. In a specific embodiment, the placenta is drained of cord blood and flushed, e.g., with perfusion solution, through the placental vasculature to remove residual blood prior to said perfusing. The perfusate is collected and processed to remove any residual erythrocytes. NK cells in the total nucleated cells in the perfusate can be isolated on the basis of expression of CD56 and CD16. In certain embodiments, the isolation of PINK cells comprises isolation using an antibody to CD56, wherein the isolated cells are CD56⁺. In another embodiment, the isolation of PINK cells comprises isolation using an antibody to CD16, wherein the isolated cells are CD16⁻. In another embodiment, the isolation of PINK cells comprises isolation using an antibody to CD56, and exclusion of a plurality of non-PINK cells using an antibody to CD16, wherein the isolated cells comprise CD56⁺, CD16⁻ cells.

Cell separation can be accomplished by any method known in the art, e.g., fluorescence-activated cell sorting (FACS), or, preferably, magnetic cell sorting using microbeads conjugated with specific antibodies. Magnetic cell separation can be performed and automated using, e.g., an AUTOMACS™ Separator (Miltenyi).

In another aspect, the placental NK cells are isolated from a plurality of placental cells. In a specific embodiment, the placental cells are, or comprise, placental perfusate cells, e.g., total nucleated cells from placental perfusate. In another specific embodiment, said plurality of placental cells is, or comprises, placental cells obtained by mechanical and/or enzymatic digestion of placental tissue. In another embodiment, said isolating is performed using one or more antibodies. In a more specific embodiment, said one or more antibodies comprises one or more of antibodies to CD3, CD16 or CD56. In a more specific embodiment, said isolating comprises isolating CD56⁺ cells from CD56⁻ cells in said plurality of placental cells. In a more specific embodiment, said isolating comprises isolating CD56⁺, CD16⁻ placental cells, e.g., placental NK cells, e.g., PINK cells, from placental cells that are CD56⁻ or CD16⁺. In a more specific embodiment, said isolating comprises isolating CD56⁺, CD16⁻, CD3⁻ placental cells from placental cells that are CD56⁻, CD16⁻, or CD3⁺. In another embodiment, said method of isolating placental NK cells results in a population of placental cells that is at least 50%, 55%, 60%, 65%, 70%, 75%, 80%, 85%, 90%, 95%, 98% or at least 99% CD56⁺, CD16⁻ NK cells.

### 5.4. Placental NK Cells from Matched Perfusate and Cord Blood

Further provided herein are NK cells obtained, and obtainable from, combinations of matched units of placental perfusate and umbilical cord blood, referred to herein as combined NK cells. “Matched units,” as used herein, indicates that the NK cells are obtained from placental perfusate cells, and umbilical cord blood cells, wherein the umbilical cord blood cells are obtained from umbilical cord blood from the placenta from which the placental perfusate is obtained, i.e., the placental perfusate cells and umbilical cord blood cells, and thus the NK cells from each, are from the same individual.

In certain embodiments, the combined placental killer cells comprise only, or substantially only, NK cells that are CD56⁺ and CD16⁻. In certain other embodiments, the combined placental killer cells comprise NK cells that are CD56⁺ and CD16⁻, and NK cells that are CD56⁺ and CD16⁺. In certain specific embodiments, the combined placental killer cells comprise at least 50%, 60%, 70%, 80%, 90%, 95%, 96%, 97%, 98%, 99% or 99.5% CD56⁺CD16⁻ NK cells (PINK cells).

In one embodiment, the combined NK cells have not been cultured. In a specific embodiment, the combined NK cells comprise a detectably higher number of CD3⁻ CD56⁺CD16⁻ NK cells than an equivalent number of NK cells from peripheral blood. In another specific embodiment, the combined NK cells comprise a detectably lower number of CD3⁻CD56⁺CD16⁻ NK cells than an equivalent number of NK cells from peripheral blood. In another specific embodiment, the combined NK cells comprise a detectably higher number of CD3⁻CD56⁺KIR2DL2/L3⁺ NK cells than an equivalent number of NK cells from peripheral blood. In another specific embodiment, the combined NK cells comprise a detectably lower number of CD3⁻CD56⁺NKp46⁺ NK cells than an equivalent number of NK cells from peripheral blood. In another specific embodiment, the combined NK cells comprise a detectably lower number of CD3⁻CD56⁺NKp30⁺ NK cells than an equivalent number of NK cells from peripheral blood. In another specific embodiment, the combined NK cells comprise a detectably lower number of CD3⁻CD56⁺2B4⁺ NK cells than an equivalent number of NK cells from peripheral blood. In another specific embodiment, the combined NK cells comprise a detectably lower number of CD3⁻CD56⁺CD94⁺ NK cells than an equivalent number of NK cells from peripheral blood.

In another embodiment, the combined NK cells have been cultured, e.g., for 21 days. In a specific embodiment, the combined NK cells comprise a detectably lower number of CD3⁻CD56⁺KIR2DL2/L3⁺ NK cells than an equivalent number of NK cells from peripheral blood. In another specific embodiment, the combined NK cells have not been cultured. In another specific embodiment, the combined NK cells comprise a detectably higher number of CD3⁻CD56⁺NKp44⁺ NK cells than an equivalent number of NK cells from peripheral blood. In a specific embodiment, the combined NK cells comprise a detectably higher number of CD3⁻CD56⁺NKp30⁺ NK cells than an equivalent number of NK cells from peripheral blood.

In another embodiment, the combined NK cells express a detectably higher amount of granzyme B than an equivalent number of peripheral blood NK cells.

Further provided herein are combinations of umbilical cord blood and combined NK cells. In various embodiments, cord blood is combined with combined NK cells at about 1×10⁴, 5×10⁴, 1×10⁵, 5×10⁵, 1×10⁶, 5×10⁶, 1×10⁷, 5×10⁷, 1×10⁸, 5×10⁸ combined NK cells per milliliter of cord blood.

### 5.5. Perfusate/Cell Combinations

In addition to placental perfusate, placental perfusate cells, combined NK cells, and placental NK cells, e.g., placental intermediate NK cells, provided herein are compositions comprising the perfusate or cells, for use in suppressing the proliferation of a tumor cell or plurality of tumor cells.

### 5.5.1. Combinations of Placental Perfusate, Perfusate Cells and Placenta-Derived Intermediate NK Cells

Further provided herein are compositions comprising combinations of the placental perfusate, placental perfusate cells, placental intermediate NK cells, and/or combined NK cells described in Sections 5.2, 5.3, or 5.4 above. In one embodiment, for example, provided herein is a volume of placental perfusate supplemented with a plurality of placental perfusate cells and/or a plurality of placental NK cells, e.g., placental intermediate NK cells, for example, obtained from placental perfusate cells or placental tissue mechanically or enzymatically disrupted. In specific embodiments, for example, each milliliter of placental perfusate is supplemented with about 1×10⁴, 5×10⁴, 1×10⁵, 5×10⁵, 1×10⁶, 5×10⁶, 1×10⁷, 5×10⁷, 1×10⁸, 5×10⁸ or more placental perfusate cells, placental intermediate NK cells, and/or combined NK cells. In another embodiment, a plurality of placental perfusate cells is supplemented with placental perfusate, placental intermediate NK cells, and/or combined NK cells. In another embodiment, a plurality of placental intermediate NK cells is supplemented with placental perfusate, placental perfusate cells, and/or combined NK cells. In certain embodiments, when perfusate is used for supplementation, the volume of perfusate is about, greater than about, or less than about, 50%, 45%, 40%, 35%, 30%, 25%, 20%, 15%, 10%, 8%, 6%, 4%, 2% or 1% of the total volume of cells (in solution) plus perfusate. In certain other embodiments, when placental perfusate cells are combined with a plurality of PINK cells and/or combined NK cells, the placental perfusate cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when PINK cells are combined with a plurality of placental perfusate cells and/or combined NK cells, the PINK cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when combined NK cells are combined with PINK cells and/or placental perfusate cells, the combined NK cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when PINK cells, combined NK cells or placental perfusate cells are used to supplement placental perfusate, the volume of solution (e.g., saline solution, culture medium or the like) in which the cells are suspended comprises about, greater than about, or less than about, 50%, 45%, 40%, 35%, 30%, 25%, 20%, 15%, 10%, 8%, 6%, 4%, 2% or 1% of the total volume of perfusate plus cells, where the PINK cells are suspended to about 1×10⁴, 5×10⁴, 1×10⁵, 5×10⁵, 1×10⁶, 5×10⁶, 1×10⁷, 5×10⁷, 1×10⁸, 5×10⁸ or more cells per milliliter prior to supplementation.

In other embodiments, any of the above combinations is, in turn, combined with umbilical cord blood or nucleated cells from umbilical cord blood.

Further provided herein is pooled placental perfusate that is obtained from two or more sources, e.g., two or more placentas, and combined, e.g., pooled. Such pooled perfusate can comprise approximately equal volumes of perfusate from each source, or can comprise different volumes from each source. The relative volumes from each source can be randomly selected, or can be based upon, e.g., a concentration or amount of one or more cellular factors, e.g., cytokines, growth factors, hormones, or the like; the number of placental cells in perfusate from each source; or other characteristics of the perfusate from each source. Perfusate from multiple perfusions of the same placenta can similarly be pooled.

Similarly, provided herein are placental perfusate cells, and placenta-derived intermediate NK cells, that are obtained from two or more sources, e.g., two or more placentas, and pooled. Such pooled cells can comprise approximately equal numbers of cells from the two or more sources, or different numbers of cells from one or more of the pooled sources. The relative numbers of cells from each source can be selected based on, e.g., the number of one or more specific cell types in the cells to be pooled, e.g., the number of CD34⁺ cells, the number of CD56⁺ cells, etc.

Pools can comprise, e.g., placental perfusate supplemented with placental perfusate cells; placental perfusate supplemented with placenta-derived intermediate NK (PINK) cells; placental perfusate supplemented with both placental perfusate cells and PINK cells; placental perfusate cells supplemented with placental perfusate; placental perfusate cells supplemented with PINK cells; placental perfusate cells supplemented with both placental perfusate and PINK cells; PINK cells supplemented with placental perfusate; PINK cells supplemented with placental perfusate cells; or PINK cells supplemented with both placental perfusate cells and placental perfusate.

Further provided herein are placental perfusate, placental perfusate cells, and placental intermediate NK cells, and pools of the same or combinations of the same, that have been assayed to determine the degree or amount of tumor suppression (that is, the potency) to be expected from, e.g., a given number of placental perfusate or PINK cells, or a given volume of perfusate. For example, an aliquot or sample number of cells is contacted with a known number of tumor cells under conditions in which the tumor cells would otherwise proliferate, and the rate of proliferation of the tumor cells in the presence of placental perfusate, perfusate cells, placental NK cells, or combinations thereof, over time (e.g., 1, 2, 3, 4, 5, 6, 7, 8, 9, or 10 weeks, or longer) is compared to the proliferation of an equivalent number of the tumor cells in the absence of perfusate, perfusate cells, placental NK cells, or combinations thereof. The potency of the placental perfusate, placental perfusate cells and/or PINK cells, or combinations or pools of the same, can be expressed, e.g., as the number of cells or volume of solution required to suppress tumor cell growth, e.g., by about 10%, 15%, 20%, 25%, 30%, 35%, 40%, 45%, 50%, or the like.

In certain embodiments, placental perfusate, placental perfusate cells, and PINK cells are provided as pharmaceutical grade administrable units. Such units can be provided in discrete volumes, e.g., 100 mL, 150 mL, 200 mL, 250 mL, 300 mL, 350 mL, 400 mL, 450 mL, 500 mL, or the like. Such units can be provided so as to contain a specified number of, e.g., placental perfusate cells, placental intermediate NK cells, or both, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. Such units can be provided to contain specified numbers of any two, or all three, of placental perfusate, placental perfusate cells, and/or PINK cells.

In the above combinations of placental perfusate, placental perfusate cells and/or PINK cells, any one, any two, or all three of the placental perfusate, placental perfusate cells and/or PINK cells can be autologous to a recipient (that is, obtained from the recipient), or homologous to a recipient (that is, obtained from at last one other individual from said recipient).

Any of the above combinations or pools of PINK cells, placental perfusate cells and/or placental perfusate can comprise CD56⁺CD16⁺ NK cells from, e.g., placental perfusate, peripheral blood, umbilical cord blood, bone marrow, or the like. In specific embodiments, the combinations comprise about, at least about, or at most about 1×10⁴, 5×10⁴, 1×10⁵, 5×10⁵, 1×10⁶, 5×10⁶ or more such NK cells per milliliter, or 1×10⁴, 5×10⁴, 1×10⁵, 5×10⁵, 1×10⁶, 5×10⁶, 1×10⁷, 5×10⁷, 1×10, 5×10⁸, 1×10⁹, 5×10⁹, 1×10¹⁰, 5×10¹⁰, 1×10¹¹ or more cells per unit. The CD56⁺CD16⁺ NK cells can be used as isolated from a natural source, or can be expanded prior to inclusion in one of the above combinations or pools. The CD56⁺CD16⁺ NK cells can be autologous (that is, obtained from the same individual as the placental perfusate, placental perfusate cells and/or PINK cells; or obtained from a recipient) or homologous (that is, derived from an individual different from the placental perfusate, placental perfusate cells and/or PINK cells; or from an individual that is not recipient).

Preferably, each unit is labeled to specify volume, number of cells, type of cells, whether the unit has been enriched for a particular type of cell, and/or potency of a given number of cells in the unit, or a given number of milliliters of the unit, causes a measurable suppression of proliferation of a particular type or types of tumor cell.

Also provided herein are compositions comprising placental intermediate NK cells, alone or in combination with placental perfusate cells and/or placental perfusate. Thus, in another aspect, provided herein is a composition comprising isolated CD56⁺, CD16⁻ NK cells, wherein said NK cells are isolated from placental perfusate, and wherein said NK cells comprise at least 50% of cells in the composition. In a specific embodiment, said NK cells comprise at least 80% of cells in the composition. In another specific embodiment, said composition comprises isolated CD56⁺, CD16⁺ NK cells. In a more specific embodiment, said CD56⁺, CD16⁺ NK cells are from a different individual than said CD56⁺, CD16⁻ NK cells. In another specific embodiment, said NK cells are from a single individual. In a more specific embodiment, said isolated NK cells comprise NK cells from at least two different individuals. In another specific embodiment, the composition comprises isolated placental perfusate. In a more specific embodiment, said placental perfusate is from the same individual as said NK cells. In another more specific embodiment, said placental perfusate comprises placental perfusate from a different individual than said NK cells. In another specific embodiment, the composition comprises placental perfusate cells. In a more specific embodiment, said placental perfusate cells are from the same individual as said NK cells. In another more specific embodiment, said placental perfusate cells are from a different individual than said NK cells. In another specific embodiment, the composition additionally comprises isolated placental perfusate and isolated placental perfusate cells, wherein said isolated perfusate and said isolated placental perfusate cells are from different individuals. In another more specific embodiment of any of the above embodiments comprising placental perfusate, said placental perfusate comprises placental perfusate from at least two individuals. In another more specific embodiment of any of the above embodiments comprising placental perfusate cells, said isolated placental perfusate cells are from at least two individuals.

### 5.5.2. Compositions Comprising Adherent Placental Stem Cells

In other embodiments, the placental perfusate, plurality of placental perfusate cells, and/or plurality of PINK cells, or a combination or pool of any of the foregoing, is supplemented with adherent placental stem cells. Such stem cells are described, e.g, in Hariri U.S. Pat. Nos. 7,045,148 and 7,255,879. Adherent placental stem cells are not trophoblasts.

The placental perfusate, plurality of placental perfusate cells, and/or plurality of PINK cells, or a combination or pool of any of the foregoing can be supplemented with, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more adherent placental cells. The adherent placental stem cells in the combinations can be, e.g., adherent placental stem cells that have been cultured for, e.g., 1, 2, 3, 4, 5, 6, 7, 8, 9, 10, 12, 14, 16, 18, 20, 22, 24, 26, 28, 30, 32, 34, 36, 38, or 40 population doublings, or more.

Adherent placental stem cells, when cultured in primary cultures or in cell culture, adhere to the tissue culture substrate, e.g., tissue culture container surface (e.g., tissue culture plastic). Adherent placental stem cells in culture assume a generally fibroblastoid, stellate appearance, with a number of cytoplasmic processes extending from the central cell body. Adherent placental stem cells are, however, morphologically distinguishable from fibroblasts cultured under the same conditions, as the placental stem cells exhibit a greater number of such processes than do fibroblasts. Morphologically, placental stem cells are also distinguishable from hematopoietic stem cells, which generally assume a more rounded, or cobblestone, morphology in culture.

Adherent placental stem cells, and populations of placental stem cells, useful in the compositions and methods provided herein, express a plurality of markers that can be used to identify and/or isolate the stem cells, or populations of cells that comprise the stem cells. The adherent placental stem cells, and adherent stem cell populations useful in the compositions and methods provided herein include stem cells and stem cell-containing cell populations obtained directly from the placenta, or any part thereof (e.g., amnion, chorion, amnion-chorion plate, placental cotyledons, umbilical cord, and the like). The adherent placental stem cell population, in one embodiment, is a population (that is, two or more) of adherent placental stem cells in culture, e.g., a population in a container, e.g., a bag.

Adherent placental stem cells generally express the markers CD73, CD 105, CD200, HLA-G, and/or OCT-4, and do not express CD34, CD38, or CD45. Adherent placental stem cells can also express HLA-ABC (MHC-1) and HLA-DR. These markers can be used to identify adherent placental stem cells, and to distinguish placental stem cells from other stem cell types. Because the placental stem cells can express CD73 and CD105, they can have mesenchymal stem cell-like characteristics. However, because the adherent placental stem cells can express CD200 and HLA-G, a fetal-specific marker, they can be distinguished from mesenchymal stem cells, e.g., bone marrow-derived mesenchymal stem cells, which express neither CD200 nor HLA-G. In the same manner, the lack of expression of CD34, CD38 and/or CD45 identifies the adherent placental stem cells as non-hematopoietic stem cells.

In one embodiment, the adherent placental stem cells are CD200⁺, HLA-G⁺, wherein the stem cells detectably suppress cancer cell proliferation or tumor growth. In a specific embodiment, said adherent stem cells are also CD73⁺ and CD105⁺. In another specific embodiment, said adherent stem cells are also CD34⁻, CD38⁻ or CD45⁻. In a more specific embodiment, said adherent stem cells are also CD34⁻, CD38⁻, CD45⁻, CD73⁺ and CD105⁺. In another embodiment, said adherent stem cells produce one or more embryoid-like bodies when cultured under conditions that allow the formation of embryoid-like bodies.

In another embodiment, the adherent placental stem cells are CD73⁺, CD105⁺, CD200⁺, wherein said stem cells detectably suppress cancer cell proliferation or tumor growth. In a specific embodiment of said populations, said adherent stem cells are HLA-G⁺. In another specific embodiment, said adherent stem cells are CD34⁻, CD38⁻ or CD45⁻. In another specific embodiment, said adherent stem cells are CD34⁻, CD38⁻ and CD45⁻. In a more specific embodiment, said adherent stem cells are CD34⁻, CD38⁻, CD45⁻, and HLA-G⁺. In another specific embodiment, said adherent placental stem cells produce one or more embryoid-like bodies when cultured under conditions that allow the formation of embryoid-like bodies.

In another embodiment, the adherent placental stem cells are CD200⁺, OCT-4⁺, wherein said stem cells detectably suppress cancer cell proliferation or tumor growth. In a specific embodiment, said adherent stem cells are CD73⁺ and CD105⁺. In another specific embodiment, said adherent stem cells are HLA-G⁺. In another specific embodiment, said adherent stem cells are CD34⁻, CD38⁻ and CD45⁻. In a more specific embodiment, said adherent stem cells are CD34⁻, CD38⁻, CD45⁻, CD73⁺, CD105⁺ and HLA-G⁺. In another specific embodiment, the adherent placental stem cells produce one or more embryoid-like bodies when cultured under conditions that allow the formation of embryoid-like bodies.

In another embodiment, the adherent placental stem cells are CD73⁺, CD105⁺ and HLA-G⁺, wherein said adherent stem cells detectably suppress cancer cell proliferation or tumor growth. In a specific embodiment of the above plurality, said adherent stem cells are also CD34⁻, CD38⁻ or CD45⁻. In another specific embodiment, said adherent stem cells are also CD34⁻, CD38⁻ and CD45⁻. In another specific embodiment, said adherent stem cells are also OCT-4⁺. In another specific embodiment, said adherent stem cells are also CD200⁺. In a more specific embodiment, said adherent stem cells are also CD34⁻, CD38⁻, CD45⁻, OCT-4⁺ and CD200⁺.

In another embodiment, the adherent placental stem cells are CD73⁺, CD105⁺ stem cells, wherein said stem cells produce one or more embryoid-like bodies under conditions that allow formation of embryoid-like bodies, and wherein said adherent stem cells detectably suppress cancer cell proliferation or tumor growth. In a specific embodiment, said adherent stem cells are also CD34⁻, CD38⁻ or CD45⁻. In another specific embodiment, said adherent stem cells are also CD34⁻, CD38⁻ and CD45⁻. In another specific embodiment, said adherent stem cells are also OCT-4⁺. In a more specific embodiment, said adherent stem cells are also OCT-4⁺, CD34⁻, CD38⁻ and CD45⁻.

In another embodiment, the adherent placental stem cells are OCT-4⁺ stem cells, wherein said adherent placental stem cells produce one or more embryoid-like bodies when cultured under conditions that allow the formation of embryoid-like bodies, and wherein said stem cells have been identified as detectably suppressing cancer cell proliferation or tumor growth.

In various embodiments, at least 10%, at least 20%, at least 30%, at least 40%, at least 50% at least 60%, at least 70%, at least 80%, at least 90%, or at least 95% of said isolated placental cells are OCT4⁺ stem cells. In a specific embodiment of the above populations, said stem cells are CD73⁺ and CD105⁺. In another specific embodiment, said stem cells are CD34⁻, CD38⁻, or CD45⁻. In another specific embodiment, said stem cells are CD200⁺. In a more specific embodiment, said stem cells are CD73⁺, CD105⁺, CD200⁺, CD34⁻, CD38⁻, and CD45⁻. In another specific embodiment, said population has been expanded, for example, passaged at least once, at least three times, at least five times, at least 10 times, at least 15 times, or at least 20 times.

In a more specific embodiment of any of the above embodiments, the adherent placental cells express ABC-p (a placenta-specific ABC transporter protein; see, e.g., Allikmets et al., Cancer Res. 58(23):5337-9 (1998)).

In another embodiment, the adherent placental stem cells are CD29⁺, CD44⁺, CD73⁺, CD90⁺, CD105⁺, CD200⁺, CD34⁻ and CD133⁻. In another embodiment, the adherent placental stem cells, the placental stem cells constitutively secrete IL-6, IL-8 and monocyte chemoattractant protein (MCP-1).

Each of the above-referenced placental stem cells can comprise placental stem cells obtained and isolated directly from a mammalian placenta, or placental stem cells that have been cultured and passaged at least 1, 2, 3, 4, 5, 6, 7, 8, 9, 10, 12, 14, 16, 18, 20, 25, 30 or more times, or a combination thereof. Tumor cell suppressive pluralities of the adherent placental stem cells described above can comprise about, at least, or no more than, 1×10⁵, 5×10⁵, 1×10⁶, 5×10⁶, 1×10⁷, 5×10⁷, 1×10, 5×10, 1×10⁹, 5×10⁹, 1×10¹⁰, 5×10¹⁰, 1×10¹¹ or more adherent placental stem cells.

### 5.5.3. Compositions Comprising Placental Stem Cell Conditioned Media

Also provided herein is the use of a tumor-suppressive composition comprising PINK cells, placental perfusate and/or placental perfusate, and additionally conditioned medium. Adherent placental stem cells, placental perfusate cells and/or placental intermediate NK cells can be used to produce conditioned medium that is tumor cell suppressive, that is, medium comprising one or more biomolecules secreted or excreted by the stem cells that have a detectable tumor cell suppressive effect on a plurality of one or more types of immune cells. In various embodiments, the conditioned medium comprises medium in which placental cells (e.g., stem cells, placental perfusate cells, PINK cells) have grown for at least 1, 2, 3, 4, 5, 6, 7, 8, 9, 10, 11, 12, 13, 14 or more days. In other embodiments, the conditioned medium comprises medium in which placental cells have grown to at least 30%, 40%, 50%, 60%, 70%, 80%, 90% confluence, or up to 100% confluence. Such conditioned medium can be used to support the culture of a separate population of placental cells, or cells of another kind. In another embodiment, the conditioned medium provided herein comprises medium in which adherent placental stem cells and non-placental stem cells have been cultured.

Such conditioned medium can be combined with any of, or any combination of, placental perfusate, placental perfusate cells, and/or placental intermediate NK cells to form a tumor cell suppressive composition. In certain embodiments, the composition comprises less than half conditioned medium by volume, e.g., about, or less than about, 50%, 45%, 40%, 35%, 30%, 25%, 20%, 15%, 10%, 5%, 4%, 3%, 2%, or 1% by volume.

Thus, in one embodiment, provided herein is a composition comprising culture medium from a culture of placental stem cells, wherein said placental stem cells (a) adhere to a substrate; (b) express CD200 and HLA-G, or express CD73, CD105, and CD200, or express CD200 and OCT-4, or express CD73, CD105, and HLA-G, or express CD73 and CD105 and facilitate the formation of one or more embryoid-like bodies in a population of placental cells that comprise the placental stem cells, when said population is cultured under conditions that allow formation of embryoid-like bodies, or express OCT-4 and facilitate the formation of one or more embryoid-like bodies in a population of placental cells that comprise the placental stem cells when said population is cultured under conditions that allow formation of embryoid-like bodies; and (c) detectably suppress the growth or proliferation of a tumor cell or population of tumor cells. In a specific embodiment, the composition further comprises a plurality of said placental stem cells. In another specific embodiment, the composition comprises a plurality of non-placental cells. In a more specific embodiment, said non-placental cells comprise CD34+ cells, e.g., hematopoietic progenitor cells, such as peripheral blood hematopoietic progenitor cells, cord blood hematopoietic progenitor cells, or placental blood hematopoietic progenitor cells. The non-placental cells can also comprise other stem cells, such as mesenchymal stem cells, e.g., bone marrow-derived mesenchymal stem cells. The non-placental cells can also be one or more types of adult cells or cell lines. In another specific embodiment, the composition comprises an anti-proliferative agent, e.g., an anti-MIP-1α or anti-MIP-1β antibody.

In a specific embodiment, placental cell-conditioned culture medium or supernatant is obtained from a plurality of placental stem cells co-cultured with a plurality of tumor cells at a ratio of about 1:1, about 2:1, about 3:1, about 4:1, or about 5:1 placental stem cells to tumor cells. For example, the conditioned culture medium or supernatant can be obtained from a culture comprising about 1×10⁵ placental stem cells, about 1×10⁶ placental stem cells, about 1×10⁷ placental stem cells, or about 1×10⁸ placental stem cells, or more. In another specific embodiment, the conditioned culture medium or supernatant is obtained from a co-culture comprising about 1×10⁵ to about 5×10⁵ placental stem cells and about 1×10⁵ tumor cells; about 1×10⁶ to about 5×10⁶ placental stem cells and about 1×10⁶ tumor cells; about 1×10⁷ to about 5×10⁷ placental stem cells and about 1×10⁷ tumor cells; or about 1×10⁸ to about 5×10⁸ placental stem cells and about 1×10⁸ tumor cells.

In a specific embodiment, the conditioned medium suitable for administration to a 70 kg individual comprises supernatant conditioned by about 70 million placental stem cells in about 200 mL culture medium.

Conditioned medium can be condensed to prepare an administrable pharmaceutical-grade product. For example, conditioned medium can be condensed to about 90%, 80%, 70%, 60%, 50%, 40%, 30%, 20%, 10% or more by removal of water, e.g., by evaporation, lyophilization, or the like. In a specific embodiment, for example, 200 mL conditioned medium from about 70 million placental stem cells can be condensed to a volume of about 180 mL, 160 mL, 140 mL, 120 mL, 100 mL, 80 mL, 60 mL, 40 mL, 20 mL or less. The conditioned medium can also be substantially dried, e.g., to a powder, e.g., by evaporation, lyophilization or the like.

### 5.6. Use of Human Placental Perfusate, Placental Perfusate Cells, PINK Cells, or Combined NK Cells, in Combination with an Antibody to Suppress Tumor Cell Growth

Provided herein are methods of suppressing the growth (e.g., proliferation) of tumor cells using human placental perfusate, placental perfusate cells, PINK cells, or combined NK cells, in combination with an antibody (e.g., an anti-GD2 antibody).

In one embodiment, provided herein is a method of suppressing the proliferation of a tumor cell, or plurality of tumor cells, comprising contacting the tumor cell or plurality of tumor cells with human placental perfusate, placental perfusate cells, combined NK cells, and/or PINK cells, in combination with an antibody, such that the proliferation of the tumor cell or plurality of tumor cells is detectably reduced compared to a tumor cell or plurality of tumor cells of the same type not contacted with the human placental perfusate, placental perfusate cells, combined NK cells, and/or PINK cells, in combination with the antibody.

As used herein, “contacting,” in one embodiment, encompasses direct physical (e.g., cell-cell) contact between a tumor cell or plurality of tumor cells and human placental perfusate, placental perfusate cells, placental NK cells (e.g., PINK cells) and/or combined NK cells, in combination with an antibody. In another embodiment, “contacting” encompasses presence in the same physical space, e.g., human placental perfusate, placental perfusate cells, placental NK cells (e.g., PINK cells), and/or combined NK cells, in combination with an antibody are placed in the same container (e.g., culture dish, multiwell plate) as a tumor cell or plurality of tumor cells. In another embodiment, “contacting” human placental perfusate, placental perfusate cells, combined NK cells, or PINK cells, and a tumor cell or plurality of tumor cells is accomplished, e.g., by injecting or infusing the placental perfusate or cells (e.g., placental perfusate cells, combined NK cells, or PINK cells) into an individual having a tumor cell or plurality of tumor cells (e.g., a cancer patient).

In certain embodiments, human placental perfusate and an antibody are used in any amount that results in a detectable therapeutic benefit to an individual comprising a tumor cell or plurality of tumor cells (e.g., a cancer patient). In certain other embodiments, human placental perfusate cells, PINK cells, and/or combined NK cells, and an antibody are used in any amount that results in a detectable therapeutic benefit to an individual comprising a tumor cell or plurality of tumor cells. Thus, in another embodiment, provided herein is a method of suppressing the proliferation of a tumor cell, or plurality of tumor cells, comprising contacting the tumor cell or plurality of tumor cells with human placental perfusate, placental perfusate cells, and/or combined NK cells, or PINK cells, in combination with an antibody, with an individual such that said contacting is detectably or demonstrably therapeutically beneficial to said individual.

As used herein, “therapeutic benefits” include, but are not limited to, e.g., reduction in the size of a tumor; lessening or cessation of expansion of a tumor; reduction in the number of cancer cells in a tissue sample, e.g., a blood sample, per unit volume; the clinical improvement in any symptom of the particular cancer said individual has, the lessening or cessation of worsening of any symptom of the particular cancer the individual has, etc. Contacting of human placental perfusate, placental perfusate cells, combined NK cells, and/or PINK cells that accomplishes any one or more of such therapeutic benefits is said to be therapeutically beneficial.

In certain embodiments, human placental perfusate cells, e.g., nucleated cells from placental perfusate, combined NK cells, and/or PINK cells are used in any amount or number that results in a detectable therapeutic benefit to an individual having a tumor cell or plurality of tumor cells (e.g., a cancer patient). Human placental perfusate cells, combined NK cells and/or placental NK cells (e.g., PINK cells) can be administered to such an individual by numbers of cells, e.g., said individual can be administered at about, at least about, or at most about, 1×10⁵, 5×10⁵, 1×10⁶, 5×10⁶, 1×10⁷, 5×10⁷, 1×10, 5×10, 1×10⁹, 5×10⁹, 1×10¹⁰, or 5×10¹⁰ human placental perfusate cells, combined NK cells and/or PINK cells. In other embodiments, human placental perfusate cells, combined NK cells, and/or PINK cells can be administered to such an individual by numbers of cells per kilogram of the individual, e.g., said individual can be administered at about, at least about, or at most about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ human placental perfusate cells, combined NK cells, and/or PINK cells per kilogram of the individual. Human placental perfusate cells, combined NK cells, and/or PINK killer cells can be administered to such an individual according to an approximate ratio between placental perfusate cells, combined NK cells, and/or PINK cells, and tumor cells in said individual. For example, human placental perfusate cells, combined NK cells, and/or PINK cells, can be administered to said individual in a ratio of about, at least about, or at most about 1:1, 1:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1 to the number of tumor cells in the individual. The number of tumor cells in such an individual can be estimated, e.g., by counting the number of tumor cells in a sample of tissue from the individual, e.g., blood sample, biopsy, or the like. In specific embodiments, e.g., for solid tumors, said counting is performed in combination with imaging of the tumor or tumors to obtain an approximate tumor volume.

Further provided herein is a method of suppressing proliferation of a tumor cell or plurality of tumor cells using human placental perfusate, placental perfusate cells, combined NK cells, and/or PINK cells, in combination with an antibody. In various embodiments, provided herein is a method of suppressing proliferation of a tumor cell or plurality of tumor cells, comprising contacting said tumor cell or tumor cells with human placental perfusate, supplemented with a plurality of placental perfusate cells or PINK cells, and an antibody; placental perfusate cells, supplemented with placental perfusate or a plurality of PINK cells, and an antibody; PINK cells, supplemented with placental perfusate or placental perfusate cells, and an antibody; a plurality of PINK cells, a plurality of combined NK cells, and an antibody; a plurality of combined NK cells, a plurality of placental perfusate cells, and an antibody; placental perfusate, supplemented with combined NK cells, and an antibody; or a combination of all of placental perfusate, placental perfusate cells, combined NK cells, and PINK cells, and an antibody.

In one specific embodiment, for example, the proliferation of a tumor cell or plurality of tumor cells is suppressed by human placental perfusate and an antibody, supplemented with a plurality of placental perfusate cells, combined NK cells, and/or a plurality of PINK cells. In specific embodiments, for example, each milliliter of human placental perfusate is supplemented with about 1×10⁴, 5×10⁴, 1×10⁵, 5×10⁵, 1×10⁶, 5×10⁶ or more placental perfusate cells or PINK cells. In other specific embodiments, placental perfusate, e.g., one unit (i.e., the collection from a single placenta), or about 100, 150, 200, 250, 300, 350, 400, 450, 500, 550, 600, 650, 700, 750, 800, 850, 900, 950 or 1000 mL of perfusate, is supplemented with about 1×10⁴, 5×10⁴, 1×10⁵, 5×10⁵, 1×10⁶, 5×10⁶, 1×10⁷, 5×10⁷, 1×10⁸, 5×10⁸ or more PINK cells, combined NK cells, and/or placental perfusate cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more PINK cells, combined NK cells, and/or placental perfusate cells.

In another specific embodiment, the proliferation of a tumor cell or plurality of tumor cells is suppressed by a plurality of placental perfusate cells and an antibody, supplemented with placental perfusate, combined NK cells, and/or PINK cells. In more specific embodiments, about 1×10⁴, 5×10⁴, 1×10⁵, 5×10⁵, 1×10⁶, 5×10⁶, 1×10⁷, 5×10⁷, 1×10⁸, 5×10⁸ or more placental perfusate cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental perfusate cells, are supplemented with about, or at least about, 1×10⁴, 5×10⁴, 1×10⁵, 5×10⁵, 1×10⁶, 5×10⁶, 1×10⁷, 5×10⁷, 1×10⁸, 5×10⁸ or more PINK cells and/or combined NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×0¹¹ or more PINK cells. In other more specific embodiments, about 1×10⁴, 5×10⁴, 1×10⁵, 5×10⁵, 1×10⁶, 5×10⁶, 1×10⁷, 5×10⁷, 1×10⁸, 5×10⁸ or more placental perfusate cells, PINK cells, and/or combined NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental perfusate cells, are supplemented with about, or at least about, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mL of perfusate, or about 1 unit of perfusate.

In another specific embodiment, the proliferation of a tumor cell or plurality of tumor cells is suppressed by a plurality of PINK cells and an antibody, supplemented by placental perfusate, placental perfusate cells, and/or combined NK cells. In more specific embodiments, about 1×10⁴, 5×10⁴, 1×10⁵, 5×10⁵, 1×10⁶, 5×10⁶, 1×10⁷, 5×10⁷, 1×10⁸, 5×10⁸ or more placental intermediate NK cells, or 1×10⁴, 5×10⁴, 1×10⁵, 5×10⁵, 1×10⁶, 5×10⁶, 1×10⁷, 5×10⁷, 1×10, 5×10⁸, 1×10⁹, 5×10⁹, 1×10¹⁰, 5×10¹⁰, 1×10¹¹ or more PINK cells, are supplemented with about 1×10⁴, 5×10⁴, 1×10⁵, 5×10⁵, 1×10⁶, 5×10⁶, 1×10⁷, 5×10⁷, 1×10⁸, 5×10⁸ or more placental perfusate cells and/or combined NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental perfusate cells and/or combined NK cells. In other more specific embodiments, about 1×10⁴, 5×10⁴, 1×10⁵, 5×10⁵, 1×10⁶, 5×10⁶, 1×10⁷, 5×10⁷, 1×10⁸, 5×10⁸ or more placental perfusate cells and/or combined NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental intermediate NK cells and/or combined NK cells are supplemented with about, or at least about, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mL of perfusate, or about 1 unit of perfusate.

In another embodiment, the proliferation of a tumor cell or plurality of tumor cells is suppressed by contacting the tumor cell or tumor cells with placental perfusate, perfusate cells, PINK cells, and/or combined NK cells, and an antibody, supplemented with adherent placental stem cells. In specific embodiments, the placental perfusate, perfusate cells, or PINK cells are supplemented with about 1×10⁴, 5×10⁴, 1×10⁵, 5×10⁵, 1×10⁶, 5×10⁶, 1×10⁷, 5×10⁷, 1×10⁸, 5×10⁸ or more adherent placental stem cells per milliliter, or 1×10⁴, 5×10⁴, 1×10⁵, 5×10⁵, 1×10⁶, 5×10⁶, 1×10⁷, 5×10⁷, 1×10, 5×10, 1×10⁹, 5×10⁹, 1×10¹⁰, 5×10¹⁰, 1×10¹¹ or more adherent placental stem cells.

In another embodiment, the proliferation of a tumor cell or plurality of tumor cells is suppressed by contacting the tumor cell or tumor cells with placental perfusate, perfusate cells, combined NK cells, and/or PINK cells, and an antibody, supplemented with adherent placental stem cell-conditioned medium, e.g., 0.1, 0.2, 0.3, 0.4, 0.5, 0.6, 0.1, 0.8, 0.9, 1, 2, 3, 4, 5, 6, 7, 8, 9, 10 mL of stem cell-conditioned culture medium per unit of perfusate, perfusate cells, combined NK cells, and/or PINK cells, or per 10⁴, 10⁵, 10⁶, 10⁷, 10⁸, 10⁹ or 10¹⁰ cells.

In other embodiments, the placental perfusate, placental perfusate cells, placental NK cells, e.g., PINK cells, combined NK cells, and combinations and pools comprising the same, are used as initially obtained, that is, perfusate as obtained during perfusion, placental perfusate cells as isolated from such perfusate, combined NK cells from such perfusate and matched umbilical cord blood, or PINK cells isolated from such perfusate or such placental perfusate cells or generated by a two-step expansion and differentiation method using CD34⁺ hematopoietic stem cells recovered from placenta. In other embodiments, the placental perfusate, placental perfusate cells, PINK cells, and combinations and pools of the same are processed prior to use. For example, placental perfusate can be used in its raw, unprocessed form as collected from the placenta. Placental perfusate can also be processed prior to use, e.g., by the negative selection of one or more types of cells, reduction in volume by dehydration; lyophilization and rehydration, etc. Similarly, populations of perfusate cells can be used as initially isolated from placental perfusate, e.g., as total nucleated cells from placental perfusate, or can be processed, e.g., to remove one or more cell types (e.g., erythrocytes). PINK cells can be used as initially isolated from placental perfusate, e.g., using CD56 microbeads, or generated by a two-step expansion and differentiation method using CD34⁺ hematopoietic stem cells recovered from placenta, or can be processed, e.g., to remove one or more non-killer cell types.

In another embodiment, provided herein is a method of suppressing the proliferation of a tumor cell or tumor cells, comprising contacting the tumor cell or tumor cells with placental perfusate, placental perfusate cells, PINK cells, combined NK cells, or pools or combinations comprising the same, and an antibody, wherein said placental perfusate, placental perfusate cells, PINK cells, combined NK cells, or pools or combinations comprising the same have been contacted with interleukin-2 (IL-2) for a period of time prior to said contacting. In certain embodiments, said period of time is about, at least, or at most 1, 2, 3, 4, 5, 6, 7, 8, 9, 10, 12, 14, 16, 18, 20, 22, 24, 26, 28, 30, 32, 34, 36, 38, 40, 42, 44, 46 or 48 prior to said contacting.

The placental perfusate, placental perfusate cells, PINK cells, combined NK cells, or pools and/or combinations of the same, and the antibody, can be administered once to an individual having cancer, or an individual having tumor cells during a course of anticancer therapy, or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy. The placental perfusate, placental perfusate cells, combined NK cells, PINK cells, pools and/or combinations of the same, and the antibody can be administered without regard to whether the placental perfusate, placental perfusate cells, combined NK cells, PINK cells, pools and/or combinations of the same, or the antibody, have been administered to a person having cancer, or having tumor cells, in the past. Thus, the methods provided herein encompasses the administration to a person having cancer or having tumor cells any combination of placental perfusate, placental perfusate cells, combined NK cells, PINK cells, pools and/or combinations comprising the same, and an antibody.

In a specific embodiment, the tumor cells are blood cancer cells. In another embodiment, the tumor cells are solid tumor cells. In various specific embodiments, the tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells. In one embodiment, the tumor cells are glioblastoma cells. In one embodiment, the tumor cells are neuroblastoma cells. In one embodiment, the tumor cells are osteosarcoma cells. In one embodiment, the tumor cells are melanoma cells. In one embodiment, the tumor cells are ovarian cancer cells. In one embodiment, the tumor cells are primary ductal carcinoma cells. In one embodiment, the tumor cells are leukemia cells. In one embodiment, the tumor cells are acute T cell leukemia cells. In one embodiment, the tumor cells are AML cells. In one embodiment, the tumor cells are CML cells. In one embodiment, the tumor cells are ALL cells. In one embodiment, the tumor cells are CLL cells. In one embodiment, the tumor cells are NHL cells. In one embodiment, the tumor cells are breast cancer cells. In one embodiment, the tumor cells are bladder cancer cells. In one embodiment, the tumor cells are Merkel cell carcinoma cells. In one embodiment, the tumor cells are head and neck cancer cells. In one embodiment, the tumor cells are lung carcinoma cells. In one embodiment, the tumor cells are colon adenocarcinoma cells. In one embodiment, the tumor cells are histiocytic lymphoma cells. In one embodiment, the tumor cells are multiple myeloma cells. In one embodiment, the tumor cells are retinoblastoma cells. In one embodiment, the tumor cells are colorectal carcinoma cells. In one embodiment, the tumor cells are colorectal adenocarcinoma cells.

### 5.7. Antibodies to be Used in the Combination Therapy with Human Placental Perfusate, Placental Perfusate Cells, PINK Cells, Combined NK Cells, or Combinations Thereof

The antibodies that can be used in the combination therapy with human placental perfusate, placental perfusate cells, PINK cells, combined NK cells, or combinations thereof, include but are not limited to individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies, as described below. In certain embodiments, an antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only one epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002).

“Polyclonal antibodies” as used herein refer to an antibody population generated in an immunogenic response to a protein having many epitopes and thus includes a variety of different antibodies directed to the same or different epitopes within the protein. Methods for producing polyclonal antibodies are known in the art (See, e.g., Short Protocols in Molecular Biology (Ausubel et al. eds., 5th ed. 2002)).

In certain embodiments of various methods provided herein, the antibody that is used in the combination therapy with human placental perfusate, placental perfusate cells, PINK cells, combined NK cells, or combinations thereof, is a monoclonal antibody. In some embodiments, the antibody that is used in the combination therapy with human placental perfusate, placental perfusate cells, PINK cells, combined NK cells, or combinations thereof, is polyclonal antibodies. In other embodiments, the antibody that is used in the combination therapy with human placental perfusate, placental perfusate cells, PINK cells, combined NK cells, or combinations thereof, is a monovalent antibody. In yet other embodiments, the antibody that is used in the combination therapy with human placental perfusate, placental perfusate cells, PINK cells, combined NK cells, or combinations thereof, is a multivalent antibody. In still other embodiments, the antibody that is used in the combination therapy with human placental perfusate, placental perfusate cells, PINK cells, combined NK cells, or combinations thereof, is a multispecific antibody. In certain embodiments, the antibody that is used in the combination therapy with human placental perfusate, placental perfusate cells, PINK cells, combined NK cells, or combinations thereof, is a single chain antibody. In some embodiments, the antibody that is used in the combination therapy with human placental perfusate, placental perfusate cells, PINK cells, combined NK cells, or combinations thereof, is a fragment of an antibody. In other embodiments, the antibody that is used in the combination therapy with human placental perfusate, placental perfusate cells, PINK cells, combined NK cells, or combinations thereof, is a scFv (e.g., including monospecific, bispecific, etc.). In yet other embodiments, the antibody that is used in the combination therapy with human placental perfusate, placental perfusate cells, PINK cells, combined NK cells, or combinations thereof, is a Fab fragment. In still other embodiments, the antibody that is used in the combination therapy with human placental perfusate, placental perfusate cells, PINK cells, combined NK cells, or combinations thereof, is a F(ab′) fragment. In certain embodiments, the antibody that is used in the combination therapy with human placental perfusate, placental perfusate cells, PINK cells, combined NK cells, or combinations thereof, is a F(ab)₂ fragment. In some embodiments, the antibody that is used in the combination therapy with human placental perfusate, placental perfusate cells, PINK cells, combined NK cells, or combinations thereof, is a F(ab′)₂ fragment. In other embodiments, the antibody that is used in the combination therapy with human placental perfusate, placental perfusate cells, PINK cells, combined NK cells, or combinations thereof, is a dsFv. In yet other embodiments, the antibody that is used in the combination therapy with human placental perfusate, placental perfusate cells, PINK cells, combined NK cells, or combinations thereof, is a Fd fragment. In still other embodiments, the antibody that is used in the combination therapy with human placental perfusate, placental perfusate cells, PINK cells, combined NK cells, or combinations thereof, is a Fv fragment. In certain embodiments, the antibody that is used in the combination therapy with human placental perfusate, placental perfusate cells, PINK cells, combined NK cells, or combinations thereof, is a diabody. In some embodiments, the antibody that is used in the combination therapy with human placental perfusate, placental perfusate cells, PINK cells, combined NK cells, or combinations thereof, is a triabody. In other embodiments, the antibody that is used in the combination therapy with human placental perfusate, placental perfusate cells, PINK cells, combined NK cells, or combinations thereof, is a tetrabody. In yet other embodiments, the antibody that is used in the combination therapy with human placental perfusate, placental perfusate cells, PINK cells, combined NK cells, or combinations thereof, is a minibody.

In one embodiment, the antibody that is used in the combination therapy with human placental perfusate, placental perfusate cells, PINK cells, combined NK cells, or combinations thereof, is an IgG1 antibody. In another embodiment, the antibody that is used in the combination therapy with human placental perfusate, placental perfusate cells, PINK cells, combined NK cells, or combinations thereof, is an IgG2 antibody. In yet another embodiment, the antibody that is used in the combination therapy with human placental perfusate, placental perfusate cells, PINK cells, combined NK cells, or combinations thereof, is an IgG3 antibody. In still another embodiment, the antibody that is used in the combination therapy with human placental perfusate, placental perfusate cells, PINK cells, combined NK cells, or combinations thereof, is an IgG4 antibody.

In one embodiment, the antibody that is used in the combination therapy with human placental perfusate, placental perfusate cells, PINK cells, combined NK cells, or combinations thereof, is a human antibody. In another embodiment, the antibody that is used in the combination therapy with human placental perfusate, placental perfusate cells, PINK cells, combined NK cells, or combinations thereof, is a humanized antibody. In yet another embodiment, the antibody that is used in the combination therapy with human placental perfusate, placental perfusate cells, PINK cells, combined NK cells, or combinations thereof, is a chimeric antibody. In yet another embodiment, the antibody that is used in the combination therapy with human placental perfusate, placental perfusate cells, PINK cells, combined NK cells, or combinations thereof, is a glycosylated antibody. In still another embodiment, the antibody that is used in the combination therapy with human placental perfusate, placental perfusate cells, PINK cells, combined NK cells, or combinations thereof, is a glycoengineered antibody.

In certain embodiments, the antibody that is used in the combination therapy with human placental perfusate, placental perfusate cells, PINK cells, combined NK cells, or combinations thereof, is a naked antibody. In some embodiments, the antibody that is used in the combination therapy with human placental perfusate, placental perfusate cells, PINK cells, combined NK cells, or combinations thereof, is a conjugated antibody (e.g., an antibody-drug conjugate (ADC)). In other embodiments, the antibody that is used in the combination therapy with human placental perfusate, placental perfusate cells, PINK cells, combined NK cells, or combinations thereof, is a radiolabeled antibody. In yet other embodiments, the antibody that is used in the combination therapy with human placental perfusate, placental perfusate cells, PINK cells, combined NK cells, or combinations thereof, is a chemolabeled antibody.

In some embodiments of various methods provided herein, the antibody that is used in the combination therapy with human placental perfusate, placental perfusate cells, PINK cells, combined NK cells, or combinations thereof, is any antibody that has anti-tumor effect (e.g., inhibiting or reducing cell growth or proliferation, inducing or increasing cell death, such as apoptosis, etc.). In certain embodiments, the antibody is selected from the group consisting of an anti-CD38 antibody, an anti-SLAMF7 antibody, an anti-CD20 antibody, an anti-GD2 antibody, an anti-HER2 antibody, an anti-PD-L1 antibody, an anti-EGFR antibody, an anti-CD52 antibody, an anti-VEGF antibody, an anti-CD19 antibody, an anti-CD30 antibody, an anti-PSMA antibody, an anti-CTLA-4 antibody, anti-PD-1 antibody, anti-VEGF receptor 2 antibody, an anti-CD22 antibody, and an anti-CD33 antibody.

In other embodiments, the antibody is selected from the group consisting of daratumumab, elotuzumab, obinutuzumab, ofatumumab, rituximab, ibritumomab, dinutuximab, trastuzumab, pertuzumab, atezolizumab, avelumab, durvalumab, cetuximab, necitumumab, panitumumab, alemtuzumab, bevacizumab, blinatumomab, brentuximab, capromab, ipilimumab, nivolumab, pembrolizumab, ramucirumab, inotuzumab, and gentuzumab.

In yet other embodiments, the antibody is a biosimilar of any one of the antibodies selected from the group consisting of daratumumab, elotuzumab, obinutuzumab, ofatumumab, rituximab, ibritumomab, dinutuximab, trastuzumab, pertuzumab, atezolizumab, avelumab, durvalumab, cetuximab, necitumumab, panitumumab, alemtuzumab, bevacizumab, blinatumomab, brentuximab, capromab, ipilimumab, nivolumab, pembrolizumab, ramucirumab, inotuzumab, and gentuzumab.

In still other embodiments, the antibody is an ADC of any one of the antibodies selected from the group consisting of daratumumab, elotuzumab, obinutuzumab, ofatumumab, rituximab, ibritumomab, dinutuximab, trastuzumab, pertuzumab, atezolizumab, avelumab, durvalumab, cetuximab, necitumumab, panitumumab, alemtuzumab, bevacizumab, blinatumomab, brentuximab, capromab, ipilimumab, nivolumab, pembrolizumab, ramucirumab, inotuzumab, and gentuzumab.

In certain embodiments of various methods provided herein, the antibody that is used in the combination therapy with human placental perfusate, placental perfusate cells, PINK cells, combined NK cells, or combinations thereof, is an anti-CD38 antibody. In some embodiments, the antibody that is used in the combination therapy is an anti-SLAMF7 antibody. In other embodiments, the antibody that is used in the combination is an anti-CD20 antibody. In yet other embodiments, the antibody that is used in the combination therapy is an anti-GD2 antibody. In still other embodiments, the antibody that is used in the combination therapy is an anti-HER2 antibody. In some embodiments, the antibody that is used in the combination therapy is an anti-PD-L1 antibody. In other embodiments, the antibody that is used in the combination therapy is an anti-EGFR antibody. In some embodiments, the antibody that is used in the combination therapy is an anti-CD52 antibody. In other embodiments, the antibody that is used in the combination is an anti-VEGF antibody. In yet other embodiments, the antibody that is used in the combination therapy is an anti-CD19 antibody. In still other embodiments, the antibody that is used in the combination therapy is an anti-CD30 antibody. In some embodiments, the antibody that is used in the combination therapy is an anti-PSMA antibody. In other embodiments, the antibody that is used in the combination therapy is an anti-CTLA-4 antibody. In some embodiments, the antibody that is used in the combination therapy is an anti-PD-1 antibody. In other embodiments, the antibody that is used in the combination is an anti-VEGF receptor 2 antibody. In yet other embodiments, the antibody that is used in the combination therapy is an anti-CD22 antibody. In still other embodiments, the antibody that is used in the combination therapy is an anti-CD33 antibody.

In one embodiment of various methods provided herein, the anti-CD38 antibody that is used in the combination therapy with human placental perfusate, placental perfusate cells, PINK cells, combined NK cells, or combinations thereof, is daratumumab. In another embodiment, the anti-SLAMF7 antibody that is used in the combination therapy is elotuzumab. In yet another embodiment, the anti-CD20 antibody that is used in the combination therapy is obinutuzumab. In still another embodiment, the anti-CD20 antibody that is used in the combination therapy is ofatuzumab. In one embodiment, the anti-CD20 antibody that is used in the combination therapy is rituximab. In one embodiment, the anti-CD20 antibody that is used in the combination therapy is ibritumomab. In another embodiment, the anti-GD2 antibody that is used in the combination therapy is dinutuximab. In yet another embodiment, the anti-HER2 antibody that is used in the combination therapy is trastuzumab. In yet another embodiment, the anti-HER2 antibody that is used in the combination therapy is pertuzumab. In still another embodiment, the anti-PD-L1 antibody that is used in the combination therapy is atezolizumab. In one embodiment, the anti-PD-L1 antibody that is used in the combination therapy is avelumab. In one embodiment, the anti-PD-L1 antibody that is used in the combination therapy is durvalumab. In another embodiment, the anti-EGFR antibody that is used in the combination therapy is cetuximab. In yet another embodiment, the anti-EGFR antibody that is used in the combination therapy is necitumumab. In still another embodiment, the anti-EGFR antibody that is used in the combination therapy is panitumumab. In one embodiment, the anti-CD52 antibody that is used in the combination therapy is alemtuzumab. In another embodiment, the anti-VEGF antibody that is used in the combination therapy is bevacizumab. In yet another embodiment, the anti-CD19 antibody that is used in the combination therapy is blinatumomab. In yet another embodiment, the anti-CD30 antibody that is used in the combination therapy is brentuximab. In still another embodiment, the anti-PSMA antibody that is used in the combination therapy is capromab. In one embodiment, the anti-CTLA-4 antibody that is used in the combination therapy is ipilimumab. In one embodiment, the anti-PD-1 antibody that is used in the combination therapy is nivolumab. In another embodiment, the anti-PD-1 antibody that is used in the combination therapy is pembrolizumab. In yet another embodiment, the anti-VEGF receptor 2 antibody that is used in the combination therapy is ramucirumab. In still another embodiment, the anti-CD22 antibody that is used in the combination therapy is inotuzumab. In still another embodiment, the anti-CD33 antibody that is used in the combination therapy is gemtuzumab.

In certain embodiments of various methods provided herein, more than one antibodies can be used in the combination therapy with human placental perfusate, placental perfusate cells, PINK cells, combined NK cells, or combinations thereof. In some embodiments, two antibodies are used in the combination therapy with human placental perfusate, placental perfusate cells, PINK cells, combined NK cells, or combinations thereof. In other embodiments, three antibodies are used in the combination therapy with human placental perfusate, placental perfusate cells, PINK cells, combined NK cells, or combinations thereof. In yet other embodiments, four antibodies are used in the combination therapy with human placental perfusate, placental perfusate cells, PINK cells, combined NK cells, or combinations thereof. In still other embodiments, five antibodies are used in the combination therapy with human placental perfusate, placental perfusate cells, PINK cells, combined NK cells, or combinations thereof.

The human placental perfusate, placental perfusate cells, PINK cells, combined NK cells, pools, and/or combinations comprising the same and the antibody can be part of an anticancer therapy regimen that further includes one or more other anticancer agents. Such anticancer agents are well-known in the art. Specific anticancer agents that may be administered to an individual having cancer, in addition to the human placental perfusate, placental perfusate cells, PINK cells, combined NK cells, pools and/or combinations of the same, include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); 0⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In some embodiments of various methods provided herein, the combination therapy of an antibody with human placental perfusate, placental perfusate cells, PINK cells, combined NK cells, or combinations thereof, further comprises a third agent. In certain embodiments, the third agent is a hematopoietic growth factor, cytokine, anti-cancer agent (e.g., a checkpoint inhibitor), antibiotic, cox-2 inhibitor, immunomodulatory agent, immunosuppressive agent, corticosteroid, or derivative thereof. In one embodiment, the third agent is IL-2. In another embodiment, the third agent is GM-CSF. In yet another embodiment, the third agent is 13-cis retinoic acid. In a specific embodiment, the antibody is an anti-GD2 antibody, and the third agent is IL-2. In another specific embodiment, the antibody is an anti-GD2 antibody, and the third agent is GM-CSF. In yet another specific embodiment, the antibody is an anti-GD2 antibody, and the third agent is 13-cis retinoic acid.

In other embodiments, the combination therapy of an antibody with human placental perfusate, placental perfusate cells, PINK cells, combined NK cells, or combinations thereof, further comprises a third agent and a fourth agent. In certain embodiments, the third or the fourth agent is a hematopoietic growth factor, cytokine, anti-cancer agent (e.g., a checkpoint inhibitor), antibiotic, cox-2 inhibitor, immunomodulatory agent, immunosuppressive agent, corticosteroid, or derivative thereof. In one embodiment, the third agent is IL-2. In another embodiment, the third agent is GM-CSF. In yet another embodiment, the third agent is 13-cis retinoic acid. In a specific embodiment, the antibody is an anti-GD2 antibody, and the third agent is IL-2. In another specific embodiment, the antibody is an anti-GD2 antibody, and the third agent is GM-CSF. In yet another specific embodiment, the antibody is an anti-GD2 antibody, and the third agent is 13-cis retinoic acid. In one embodiment, the third agent is IL-2, and the fourth agent is GM-CSF. In another embodiment, the third agent is IL-2, and the fourth agent is 13-cis retinoic acid. In yet another embodiment, the third agent is GM-CSF, and the fourth agent is 13-cis retinoic acid. In one embodiment, the antibody is an anti-GD2 antibody, the third agent is IL-2, and the fourth agent is GM-CSF. In another embodiment, the antibody is an anti-GD2 antibody, the third agent is IL-2, and the fourth agent is 13-cis retinoic acid. In yet another embodiment, the antibody is an anti-GD2 antibody, the third agent is GM-CSF, and the fourth agent is 13-cis retinoic acid.

In other embodiments, the combination therapy of an antibody with human placental perfusate, placental perfusate cells, PINK cells, combined NK cells, or combinations thereof, further comprises a third agent, a fourth agent, and a fifth agent. In certain embodiments, the third, the fourth, or the fifth agent is a hematopoietic growth factor, cytokine, anti-cancer agent (e.g., a checkpoint inhibitor), antibiotic, cox-2 inhibitor, immunomodulatory agent, immunosuppressive agent, corticosteroid, or derivative thereof. In one embodiment, the third agent is IL-2. In another embodiment, the third agent is GM-CSF. In yet another embodiment, the third agent is 13-cis retinoic acid. In a specific embodiment, the antibody is an anti-GD2 antibody, and the third agent is IL-2. In another specific embodiment, the antibody is an anti-GD2 antibody, and the third agent is GM-CSF. In yet another specific embodiment, the antibody is an anti-GD2 antibody, and the third agent is 13-cis retinoic acid. In one embodiment, the third agent is IL-2, and the fourth agent is GM-CSF. In another embodiment, the third agent is IL-2, and the fourth agent is 13-cis retinoic acid. In yet another embodiment, the third agent is GM-CSF, and the fourth agent is 13-cis retinoic acid. In one embodiment, the antibody is an anti-GD2 antibody, the third agent is IL-2, and the fourth agent is GM-CSF. In another embodiment, the antibody is an anti-GD2 antibody, the third agent is IL-2, and the fourth agent is 13-cis retinoic acid. In yet another embodiment, the antibody is an anti-GD2 antibody, the third agent is GM-CSF, and the fourth agent is 13-cis retinoic acid. In still another embodiment, the third agent is IL-2, the fourth agent is GM-CSF, and the fifth agent is 13-cis retinoic acid. In still another embodiment, the antibody is an anti-GD2 antibody, the third agent is IL-2, the fourth agent is GM-CSF, and the fifth agent is 13-cis retinoic acid.

### 5.8. Treatment of NK Cells with an Immunomodulatory Compound

Isolated NK cells, e.g., PINK cells or combined NK cells, as described elsewhere herein, can be treated with an immunomodulatory compound, e.g., contacted with an immunomodulatory compound, to enhance the antitumor activity of the cell. Thus, provided herein is a method of increasing the cytotoxicity of a NK cell against a tumor cell comprising contacting the NK cell with an immunomodulatory compound for a time and in a concentration sufficient for the NK cell to demonstrate increased cytotoxicity towards a tumor cell compared to a NK cell not contacted with the immunomodulatory compound. In another embodiment, provided herein is a method of increasing the expression of granzyme B in a NK cell comprising contacting the NK cell with an immunomodulatory compound for a time and in a concentration sufficient for the NK cell to demonstrate increased expression of granzyme B compared to a NK cell not contacted with the immunomodulatory compound. The immunomodulatory compound can be any immunomodulatory compound described below, e.g., lenalidomide, pomalidomide, or thalidomide.

Also provided herein is a method of increasing the cycotoxicity of a population of NK cells, e.g., PINK cells or combined NK cells, to a plurality of tumor cells comprising contacting the population of NK cells with an immunomodulatory compound for a time and in a concentration sufficient for the population of NK cells to demonstrate detectably increased cytotoxicity towards said plurality of tumor cells compared to an equivalent number of NK cells not contacted with the immunomodulatory compound. In another embodiment, provided herein is a method of increasing the expression of granzyme B in a population of NK cells comprising contacting the population of NK cells with an immunomodulatory compound for a time and in a concentration sufficient for the population of NK cells to express a detectably increased amount of granzyme B compared to an equivalent number of NK cells not contacted with the immunomodulatory compound. In a specific embodiment, said population of NK cells is contained within placental perfusate cells, e.g., total nucleated cells from placental perfusate.

In specific embodiments of the above embodiments, the NK cells are CD56⁺, CD16 PINK cells. In another specific embodiment of the above embodiments, the NK cells are combined NK cells, i.e., NK cells from matched placental perfusate and umbilical cord blood.

In another specific embodiment, said plurality of NK cells, e.g., PINK cells or combined NK cells, contacted with said immunomodulatory compound express one or more of BAX, CCL5, CCR5, CSF2, FAS, GUSB, IL2RA, or TNFRSF18 at a higher level than an equivalent number of said NK cells not contacted with said immunomodulatory compound. In another specific embodiment, said plurality of NK cells, e.g., PINK cells, contacted with said immunomodulatory compound express one or more of ACTB, BAX, CCL2, CCL3, CCL5, CCR5, CSF1, CSF2, ECE1, FAS, GNLY, GUSB, GZMB, IL1A, IL2RA, IL8, IL10, LTA, PRF1, PTGS2, SKI, and TBX21 at a higher level than an equivalent number of said NK cells not contacted with said immunomodulatory compound.

Also provided herein is a method of increasing the cycotoxicity of a population of human placental perfusate cells, e.g., total nucleated cells from placental perfusate, towards a plurality of tumor cells, comprising contacting the placental perfusate cells with an immunomodulatory compound for a time and in a concentration sufficient for the placental perfusate cells to demonstrate detectably increased cytotoxicity towards said plurality of tumor cells compared to an equivalent number of placental perfusate cells not contacted with the immunomodulatory compound. In another embodiment, provided herein is a method of increasing the expression of granzyme B in a population of placental perfusate cells comprising contacting the population of placental perfusate cells with an immunomodulatory compound for a time and in a concentration sufficient for the population of placental perfusate cells to express a detectably increased amount of granzyme B compared to an equivalent number of placental perfusate cells not contacted with the immunomodulatory compound.

Immunomodulatory compounds can either be commercially purchased or prepared according to the methods described in the patents or patent publications referred to herein, all of which are incorporated by reference. Further, optically pure compositions can be asymmetrically synthesized or resolved using known resolving agents or chiral columns as well as other standard synthetic organic chemistry techniques. Immunomodulatory compounds may be racemic, stereomerically enriched or stereomerically pure, and may encompass pharmaceutically acceptable salts, solvates, and prodrugs thereof.

As used herein and unless otherwise indicated, the terms “immunomodulatory compounds” encompass small organic molecules that markedly inhibit TNF-α, LPS induced monocyte IL-1B and IL-12, and partially inhibit IL-6 production. In specific examples, the immunomodulatory compounds are lenalidomide, pomalidomide or thalidomide.

Specific examples of immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), including, but not limited to, those disclosed in U.S. Pat. Nos. 5,635,517, 6,476,052, 6,555,554, and 6,403,613; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; and isoindole-imide compounds such as those described in U.S. patent publication no. 2003/0045552 A1, U.S. Pat. No. 7,091,353, and WO 02/059106. The entireties of each of the patents and patent applications identified herein are incorporated herein by reference. Immunomodulatory compounds do not include thalidomide.

In certain embodiments, the immunomodulatory compounds are 1-oxo- and 1,3 dioxo-2-(2,6-dioxopiperidin-3-yl) isoindolines substituted with amino in the benzo ring as described in U.S. Pat. No. 5,635,517, which is incorporated herein by reference in its entirety. These compounds have the structure I:

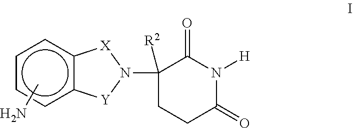

in which one of X and Y is C═O, the other of X and Y is C═O or CH₂, and R² is hydrogen or lower alkyl, in particular methyl. Specific immunomodulatory compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2,6-dioxopiperidin-3-yl)-4-aminoisoindoline; and
1,3-dioxo-2-(2,6-dioxopiperidin-3-yl)-5-aminoisoindoline.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and WO 98/03502, each of which is incorporated herein by reference. Representative compounds are of formula:

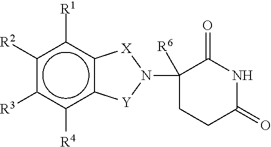

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R²,

R³, and R⁴ is fluoro or (ii) one of R¹, R², R³, or R⁴ is amino.

Compounds representative of this class are of the formulas:

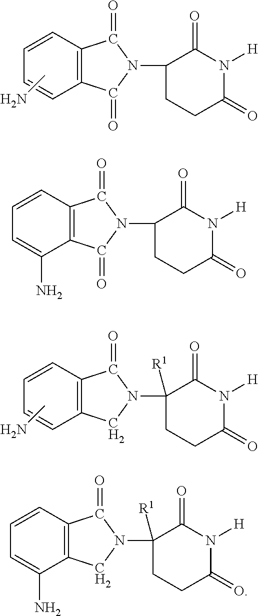

wherein R¹ is hydrogen or methyl. In a separate embodiment, encompassed is the use of enantiomerically pure forms (e.g. optically pure (R) or (S) enantiomers) of these compounds.

Still other specific immunomodulatory compounds belong to a class of isoindole-imides disclosed in U.S. Patent Application Publication Nos. US 2003/0096841 and US 2003/0045552, and WO 02/059106, each of which are incorporated herein by reference. Representative compounds are of formula II:

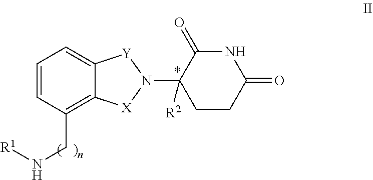

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₈)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₈)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₈)heteroaryl;

R⁵ is (C₁-C₈)alkyl, (C₂-C₈)alkenyl, (C₂-C₈)alkynyl, benzyl, aryl, or (C₂-C₈)heteroaryl;

each occurrence of R⁶ is independently H, (C₁-C₈)alkyl, (C₂-C₈)alkenyl, (C₂-C₈)alkynyl, benzyl, aryl, (C₂-C₈)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₈)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₈)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

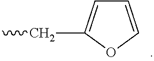

In another embodiment of the compounds of formula II, R¹ is

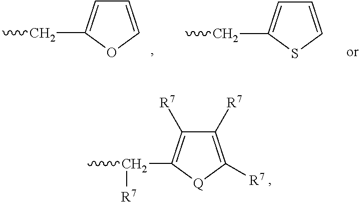

wherein Q is O or S, and each occurrence of R⁷ is independently H, (C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₈)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₈)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl} cyclopropyl-carboxamide; 2-chloro-N-{(2-(2,6-dioxo(3-piperidyl))-1,3-dioxoisoindolin-4-yl)methyl} acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl} piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl} methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl} (octylamino)carboxamide; and N-{[2-(2,6-dioxo(3-piperidyl))-1,3-dioxoisoindolin-4-yl] methyl}(benzylamino)carboxamide.

Still other specific immunomodulatory compounds belong to a class of isoindole-imides disclosed in U.S. Patent Application Publication No. 2002/0045643, International Publication No. WO 98/54170, and U.S. Pat. No. 6,395,754, each of which is incorporated herein by reference. Representative compounds are of formula III:

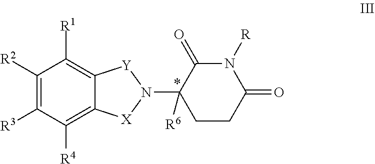

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4; each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center.

Other representative compounds are of formula:

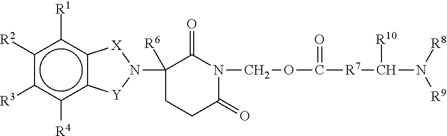

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—;

R¹⁰ is hydrogen, alkyl of to 8 carbon atoms, or phenyl.

Other representative compounds are of formula:

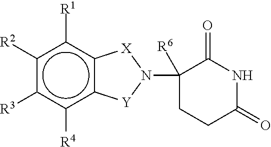

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

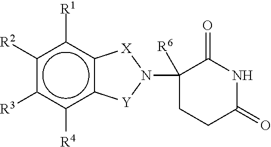

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

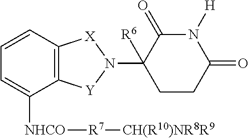

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

The most preferred immunomodulatory compounds are 4-(amino)-2-(2,6-dioxo(3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). The compounds are available from Celgene Corporation, Warren, N.J. 4-(Amino)-2-(2,6-dioxo(3-piperidyl))-isoindoline-1,3-dione has the following chemical structure:

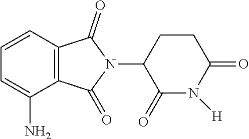 [SMILES: Nc1cccc2c1C(=O)N(C1CCC(=O)NC1=O)C2=O]

The compound 3-(4-amino-1-oxo-1,3-dihydro-isoindol-2-yl)-piperidine-2,6-dione has the following chemical structure:

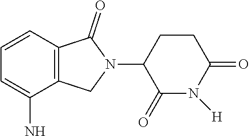 [SMILES: Nc1cccc2c1CN(C1CCC(=O)NC1=O)C2=O]

In another embodiment, specific immunomodulatory compounds encompass polymorphic forms of 3-(4-amino-1-oxo-1,3 dihydro-isoindol-2-yl)-piperidene-2,6-dione such as Form A, B, C, D, E, F, G and H, disclosed in U.S. publication no. US 2005/0096351 A1, which is incorporated herein by reference. For example, Form A of 3-(4-amino-1-oxo-1,3 dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 2θ, and has a differential scanning calorimetry melting temperature maximum of about 270° C. Form A is weakly or not hygroscopic and appears to be the most thermodynamically stable anhydrous polymorph of 3-(4-amino-1-oxo-1,3 dihydro-isoindol-2-yl)-piperidine-2,6-dione discovered thus far.

Form B of 3-(4-amino-1-oxo-1,3 dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 2θ, and has endotherms from DSC curve of about 146 and 268° C., which are identified dehydration and melting by hot stage microscopy experiments. Interconversion studies show that Form B converts to Form E in aqueous solvent systems, and converts to other forms in acetone and other anhydrous systems.

Form C of 3-(4-amino-1-oxo-1,3 dihydro-isoindol-2-yl)-piperidene-2,6-dione is a hemisolvated crystalline material that can be obtained from solvents such as, but not limited to, acetone. Form C has an X-ray powder diffraction pattern comprising significant peaks at approximately 15.5 and 25 degrees 2θ, and has a differential scanning calorimetry melting temperature maximum of about 269° C. Form C is not hygroscopic below about 85% RH, but can convert to Form B at higher relative humidities.

Form D of 3-(4-amino-1-oxo-1,3 dihydro-isoindol-2-yl)-piperidene-2,6-dione is a crystalline, solvated polymorph prepared from a mixture of acetonitrile and water. Form D has an X-ray powder diffraction pattern comprising significant peaks at approximately 27 and 28 degrees 2θ, and has a differential scanning calorimetry melting temperature maximum of about 270° C. Form D is either weakly or not hygroscopic, but will typically convert to Form B when stressed at higher relative humidities.

Form E of 3-(4-amino-1-oxo-1,3 dihydro-isoindol-2-yl)-piperidene-2,6-dione is a dihydrated, crystalline material that can be obtained by slurrying 3-(4-amino-1-oxo-1,3 dihydro-isoindol-2-yl)-piperidene-2,6-dione in water and by a slow evaporation of 3-(4-amino-1-oxo-1,3 dihydro-isoindol-2-yl)-piperidene-2,6-dione in a solvent system with a ratio of about 9:1 acetone:water. Form E has an X-ray powder diffraction pattern comprising significant peaks at approximately 20, 24.5 and 29 degrees 2θ, and has a differential scanning calorimetry melting temperature maximum of about 269° C. Form E can convert to Form C in an acetone solvent system and to Form G in a THF solvent system. In aqueous solvent systems, Form E appears to be the most stable form. Desolvation experiments performed on Form E show that upon heating at about 125° C. for about five minutes, Form E can convert to Form B. Upon heating at 175° C. for about five minutes, Form B can convert to Form F.

Form F of 3-(4-amino-1-oxo-1,3 dihydro-isoindol-2-yl)-piperidene-2,6-dione is an unsolvated, crystalline material that can be obtained from the dehydration of Form E. Form F has an X-ray powder diffraction pattern comprising significant peaks at approximately 19, 19.5 and 25 degrees 2θ, and has a differential scanning calorimetry melting temperature maximum of about 269° C.

Form G of 3-(4-amino-1-oxo-1,3 dihydro-isoindol-2-yl)-piperidene-2,6-dione is an unsolvated, crystalline material that can be obtained from slurrying forms B and E in a solvent such as, but not limited to, tetrahydrofuran (THF). Form G has an X-ray powder diffraction pattern comprising significant peaks at approximately 21, 23 and 24.5 degrees 2θ, and has a differential scanning calorimetry melting temperature maximum of about 267° C.

Form H of 3-(4-amino-1-oxo-1,3 dihydro-isoindol-2-yl)-piperidene-2,6-dione is a partially hydrated (about 0.25 moles) crystalline material that can be obtained by exposing Form E to 0% relative humidity. Form H has an X-ray powder diffraction pattern comprising significant peaks at approximately 15, 26 and 31 degrees 2θ, and has a differential scanning calorimetry melting temperature maximum of about 269° C.

Other specific immunomodulatory compounds usable in the methods provided herein include, but are not limited to, 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

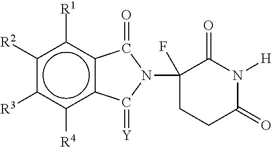

wherein Y is oxygen or H² and
each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds usable in the methods provided herein include, but are not limited to, the tetra substituted 2-(2,6-dioxopiperdin-3-yl)-1-oxoisoindolines described in U.S. Pat. No. 5,798,368, which is incorporated herein by reference. Representative compounds are of formula:

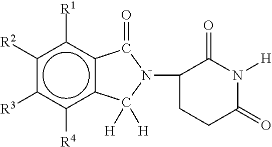

wherein each of R¹, R², R³, and R⁴, independently of the others, is halo, alkyl of 1 to 4 carbon atoms, or alkoxy of 1 to 4 carbon atoms.

Other specific immunomodulatory compounds that can be used in the methods provided herein include, but are not limited to, 1-oxo and 1,3-dioxo-2-(2,6-dioxopiperidin-3-yl) isoindolines disclosed in U.S. Pat. No. 6,403,613, which is incorporated herein by reference. Representative compounds are of formula:

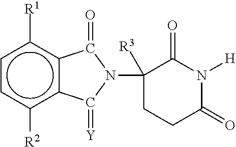

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

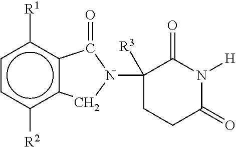

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other compounds that can be used in the methods provided herein are of formula:

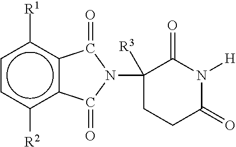

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl.

Other specific immunomodulatory compounds that can be used in the methods provided herein include, but are not limited to, 1-oxo and 1,3-dioxoisoindolines substituted in the 4- or 5-position of the indoline ring described in U.S. Pat. No. 6,380,239 and U.S. Application Publication No. 2006/0084815, which are incorporated herein by reference. Representative compounds are of formula:

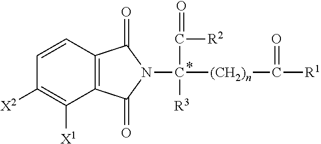

in which the carbon atom designated C* constitutes a center of chirality (when n is not zero and R¹ is not the same as R²); one of X¹ and X² is amino, nitro, alkyl of one to six carbons, or NH—Z, and the other of X¹ or X² is hydrogen; each of R¹ and R² independent of the other, is hydroxy or NH—Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further compounds that can be used in the methods provided herein are of formula:

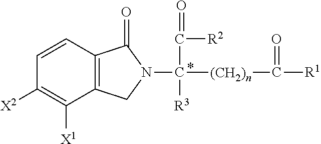

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl or an alkyl or acyl of one to six carbons; and n has a value of 0, 1, or 2.

Specific examples of compounds that can be used in the methods provided herein include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-carbamoyl-butyric acid, which have the following structures, respectively, and pharmaceutically acceptable salts, solvates, prodrugs, and stereoisomers thereof:

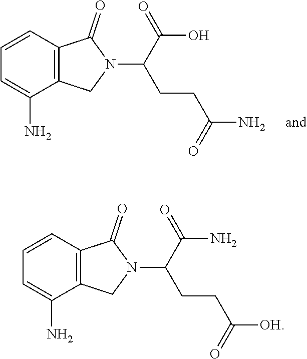 [SMILES: NC(=O)CCC(C(=O)O)N1Cc2c(N)cccc2C1=O ; "and" ; NC(=O)C(CCC(=O)O)N1Cc2c(N)cccc2C1=O]

Other representative compounds are of formula:

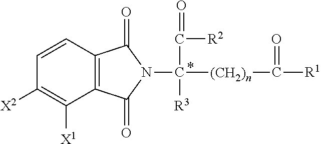

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

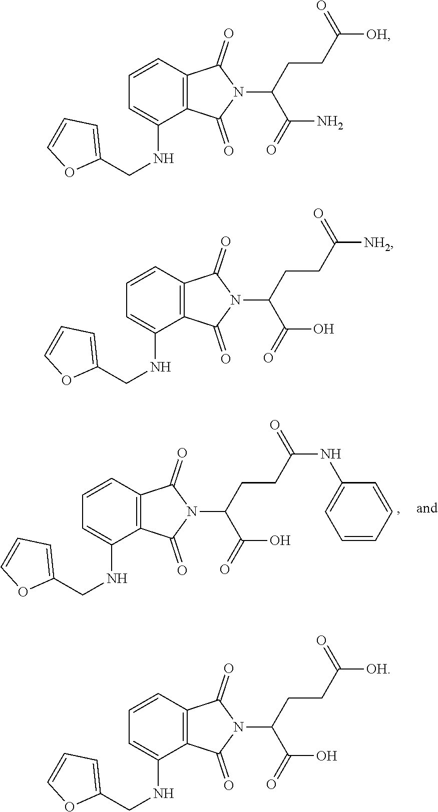 [SMILES: NC(=O)C(CCC(=O)O)N1C(=O)c2cccc(NCc3ccco3)c2C1=O ; NC(=O)CCC(C(=O)O)N1C(=O)c2cccc(NCc3ccco3)c2C1=O ; O=C(CCC(C(=O)O)N1C(=O)c2cccc(NCc3ccco3)c2C1=O)Nc1ccccc1 ; O=C(O)CCC(C(=O)O)N1C(=O)c2cccc(NCc3ccco3)c2C1=O]

Other specific examples of the compounds are of formula:

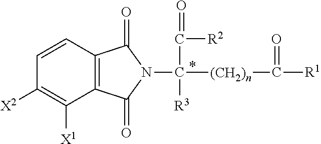

wherein one of X¹ and X² is nitro, or NH—Z, and the other of X¹ or X² is hydrogen;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2;

provided that if one of X¹ and X² is nitro, and n is 1 or 2, then R¹ and R² are other than hydroxy; and

if —COR² and —(CH₂)ₙCOR¹ are different, the carbon atom designated C* constitutes a center of chirality.

Other representative compounds are of formula:

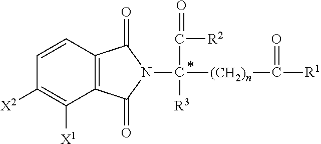

wherein one of X¹ and X² is alkyl of one to six carbons;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

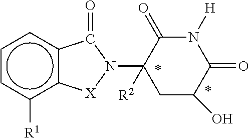

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

Compounds provided herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

Various immunomodulatory compounds contain one or more chiral centers, and can exist as racemic mixtures of enantiomers or mixtures of diastereomers. Encompassed is the use of stereomerically pure forms of such compounds, as well as the use of mixtures of those forms. For example, mixtures comprising equal or unequal amounts of the enantiomers of a particular immunomodulatory compounds may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.9. Administration of Human Placental Perfusate, Placental Perfusate Cells, PINK Cells, Combined NK Cells, or Combinations Thereof in Combination with an Antibody

The human placental perfusate, human placental perfusate cells, PINK cells, combined NK cells, populations of cells comprising such cells, or combinations thereof, and the antibody can be administered to an individual, e.g., an individual having tumor cells, e.g., a cancer patient, concurrently or sequentially. In certain embodiments, the human placental perfusate, human placental perfusate cells, PINK cells, combined NK cells, populations of cells comprising such cells, or combinations thereof, and the antibody are administered concurrently. In other embodiments, the human placental perfusate, human placental perfusate cells, PINK cells, combined NK cells, populations of cells comprising such cells, or combinations thereof, and the antibody are administered sequentially. In one embodiment, the human placental perfusate, human placental perfusate cells, PINK cells, combined NK cells, populations of cells comprising such cells, or combinations thereof, are administered before the antibody. In another embodiment, the human placental perfusate, human placental perfusate cells, PINK cells, combined NK cells, populations of cells comprising such cells, or combinations thereof, are administered after the antibody.

The human placental perfusate, human placental perfusate cells, PINK cells, combined NK cells, populations of cells comprising such cells, or combinations thereof, and the antibody may be administered via the same or different routes of administration. In certain embodiments, the human placental perfusate, human placental perfusate cells, PINK cells, combined NK cells, populations of cells comprising such cells, or combinations thereof, and the antibody are administered via the same route of administration. In other embodiments, the human placental perfusate, human placental perfusate cells, PINK cells, combined NK cells, populations of cells comprising such cells, or combinations thereof, and the antibody are administered via different routes of administration.

The human placental perfusate, human placental perfusate cells, PINK cells, combined NK cells, populations of cells comprising such cells, or combinations thereof, can be administered to an individual, e.g., an individual having tumor cells, e.g., a cancer patient, by any medically-acceptable route known in the art suitable to the administration of live cells. In various embodiments, the cells provided herein can be surgically implanted, injected, infused, e.g., by way of a catheter or syringe, or otherwise administered directly or indirectly to the site in need of repair or augmentation. In one embodiment, the cells are administered to an individual intravenously. In another embodiment, the cells are administered to an individual intracranially. In yet another embodiment, the cells are administered to an individual intrathecally. In another embodiment, the cells are administered to the individual at the site of a tumor, e.g., a solid tumor. In a specific embodiment in which the individual has a tumor at more than one site, the cells are administered to at least two, or all, tumor sites. In certain other embodiments, the cells provided herein, or compositions comprising the cells, are administered orally, nasally, intraarterially, parenterally, ophthalmically, intramuscularly, subcutaneously, intraperitoneally, intracerebrally, intraventricularly, intracerebroventricularly, intracistemally, intraspinally and/or perispinally. In certain specific embodiments, the cells are delivered via intracranial or intravertebral needles and/or catheters with or without pump devices.

The antibody can be administered to an individual, e.g., an individual having tumor cells, e.g., a cancer patient, by any medically-acceptable route known in the art suitable to the administration of antibody. In various embodiments, the antibody provided herein can be surgically implanted, injected, infused, e.g., by way of a catheter or syringe, or otherwise administered directly or indirectly to the site in need of repair or augmentation. In one embodiment, the antibody is administered to an individual intravenously. In another embodiment, the antibody is administered to an individual intracranially. In yet another embodiment, the antibody is administered to an individual intrathecally. In another embodiment, the antibody is administered to the individual at the site of a tumor, e.g., a solid tumor. In a specific embodiment in which the individual has a tumor at more than one site, the antibody is administered to at least two, or all, tumor sites. In certain other embodiments, the antibody is administered orally, nasally, intraarterially, parenterally, ophthalmically, intramuscularly, subcutaneously, intraperitoneally, intracerebrally, intraventricularly, intracerebroventricularly, intracistemally, intraspinally and/or perispinally. In certain specific embodiments, the antibody is delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, and the antibody are administered to an individual intravenously. In some embodiments, the human placental perfusate, human placental perfusate cells, PINK cells, combined NK cells, populations of cells comprising such cells, or combinations thereof, and the antibody are administered to an individual intracranially. In other embodiments, the human placental perfusate, human placental perfusate cells, PINK cells, combined NK cells, populations of cells comprising such cells, or combinations thereof, and the antibody are administered to an individual intrathecally. In certain other embodiments, the human placental perfusate, human placental perfusate cells, PINK cells, combined NK cells, populations of cells comprising such cells, or combinations thereof, and the antibody are administered orally, nasally, intraarterially, parenterally, ophthalmically, intramuscularly, subcutaneously, intraperitoneally, intracerebrally, intraventricularly, intracerebroventricularly, intracistemally, intraspinally and/or perispinally. In various embodiments, the human placental perfusate, human placental perfusate cells, PINK cells, combined NK cells, populations of cells comprising such cells, or combinations thereof, and the antibody are surgically implanted, injected, infused, e.g., by way of a catheter or syringe, or otherwise administered directly or indirectly to the site in need of repair or augmentation. In certain specific embodiments, the human placental perfusate, human placental perfusate cells, PINK cells, combined NK cells, populations of cells comprising such cells, or combinations thereof, and the antibody are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and the antibody is administered intracranially to an individual. In some embodiments, the human placental perfusate, human placental perfusate cells, PINK cells, combined NK cells, populations of cells comprising such cells, or combinations thereof, are administered intracranially, and the antibody is administered intrathecally to an individual. In other embodiments, the human placental perfusate, human placental perfusate cells, PINK cells, combined NK cells, populations of cells comprising such cells, or combinations thereof, are administered intrathecally, and the antibody is administered intravenously to an individual. In certain embodiments of various methods provided herein, the human placental perfusate, human placental perfusate cells, PINK cells, combined NK cells, populations of cells comprising such cells, or combinations thereof, are administered intravenously, and the antibody is administered intrathecally to an individual. In some embodiments, the human placental perfusate, human placental perfusate cells, PINK cells, combined NK cells, populations of cells comprising such cells, or combinations thereof, are administered intracranially, and the antibody is administered intravenously to an individual. In other embodiments, the human placental perfusate, human placental perfusate cells, PINK cells, combined NK cells, populations of cells comprising such cells, or combinations thereof, are administered intrathecally, and the antibody is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and the antibody is administered by an implanted catheter to an individual. In some embodiments, the human placental perfusate, human placental perfusate cells, PINK cells, combined NK cells, populations of cells comprising such cells, or combinations thereof, are administered intracranially, and the antibody is administered an implanted catheter to an individual. In other embodiments, the human placental perfusate, human placental perfusate cells, PINK cells, combined NK cells, populations of cells comprising such cells, or combinations thereof, are administered intrathecally, and the antibody is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and the antibody is administered intrathecally to an individual. In some embodiments, the human placental perfusate, human placental perfusate cells, PINK cells, combined NK cells, populations of cells comprising such cells, or combinations thereof, are administered an implanted catheter, and the antibody is administered intravenously to an individual. In other embodiments, the human placental perfusate, human placental perfusate cells, PINK cells, combined NK cells, populations of cells comprising such cells, or combinations thereof, are administered an implanted catheter, and the antibody is administered intracranially to an individual.

In certain embodiments, the anti-CD38 antibody and the human placental perfusate, placental perfusate cells, PINK cells, combined NK cells, or combinations thereof are administered concurrently. In other embodiments, the anti-CD38 antibody is administered before administration of the human placental perfusate, placental perfusate cells, PINK cells, combined NK cells, or combinations thereof. In yet other embodiments, the anti-CD38 antibody is administered after administration of the human placental perfusate, placental perfusate cells, PINK cells, combined NK cells, or combinations thereof. In one embodiment, the anti-CD38 antibody is daratumumab. In certain embodiments, daratumumab and the human placental perfusate, placental perfusate cells, PINK cells, combined NK cells, or combinations thereof are administered concurrently. In other embodiments, daratumumab is administered before administration of the human placental perfusate, placental perfusate cells, PINK cells, combined NK cells, or combinations thereof. In yet other embodiments, daratumumab is administered after administration of the human placental perfusate, placental perfusate cells, PINK cells, combined NK cells, or combinations thereof. In certain embodiments, daratumumab and the human placental perfusate, placental perfusate cells, PINK cells, combined NK cells, or combinations thereof are administered via the same route of administration. In other embodiments, daratumumab and the human placental perfusate, placental perfusate cells, PINK cells, combined NK cells, or combinations thereof are administered via different routes of administration. In certain embodiments, daratumumab and the human placental perfusate, placental perfusate cells, PINK cells, combined NK cells, or combinations thereof are administered intravenously. In other embodiments, daratumumab and the human placental perfusate, placental perfusate cells, PINK cells, combined NK cells, or combinations thereof are administered intracranially. In yet other embodiments, daratumumab and the human placental perfusate, placental perfusate cells, PINK cells, combined NK cells, or combinations thereof are administered intrathecally. In still other embodiments, daratumumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, daratumumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and daratumumab is administered intracranially to an individual. In some embodiments, the human placental perfusate, human placental perfusate cells, PINK cells, combined NK cells, populations of cells comprising such cells, or combinations thereof, are administered intracranially, and daratumumab is administered intrathecally to an individual. In other embodiments, the human placental perfusate, human placental perfusate cells, PINK cells, combined NK cells, populations of cells comprising such cells, or combinations thereof, are administered intrathecally, and daratumumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and daratumumab is administered intrathecally to an individual. In some embodiments, the human placental perfusate, human placental perfusate cells, PINK cells, combined NK cells, populations of cells comprising such cells, or combinations thereof, are administered intracranially, and daratumumab is administered intravenously to an individual. In other embodiments, the human placental perfusate, human placental perfusate cells, PINK cells, combined NK cells, populations of cells comprising such cells, or combinations thereof, are administered intrathecally, and daratumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and daratumumab is administered by an implanted catheter to an individual. In some embodiments, the human placental perfusate, human placental perfusate cells, PINK cells, combined NK cells, populations of cells comprising such cells, or combinations thereof, are administered intracranially, and daratumumab is administered an implanted catheter to an individual. In other embodiments, the human placental perfusate, human placental perfusate cells, PINK cells, combined NK cells, populations of cells comprising such cells, or combinations thereof, are administered intrathecally, and daratumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and daratumumab is administered intrathecally to an individual. In some embodiments, the human placental perfusate, human placental perfusate cells, PINK cells, combined NK cells, populations of cells comprising such cells, or combinations thereof, are administered an implanted catheter, and daratumumab is administered intravenously to an individual. In other embodiments, the human placental perfusate, human placental perfusate cells, PINK cells, combined NK cells, populations of cells comprising such cells, or combinations thereof, are administered an implanted catheter, and daratumumab is administered intracranially to an individual.

In certain embodiments, the anti-SLAMF7 antibody and the human placental perfusate, placental perfusate cells, PINK cells, combined NK cells, or combinations thereof are administered concurrently. In other embodiments, the anti-SLAMF7 antibody is administered before administration of the human placental perfusate, placental perfusate cells, PINK cells, combined NK cells, or combinations thereof. In yet other embodiments, the anti-SLAMF7 antibody is administered after administration of the human placental perfusate, placental perfusate cells, PINK cells, combined NK cells, or combinations thereof. In one embodiment, the anti-SLAMF7 antibody is elotuzumab. In certain embodiments, elotuzumab and the human placental perfusate, placental perfusate cells, PINK cells, combined NK cells, or combinations thereof are administered concurrently. In other embodiments, elotuzumab is administered before administration of the human placental perfusate, placental perfusate cells, PINK cells, combined NK cells, or combinations thereof. In yet other embodiments, elotuzumab is administered after administration of the human placental perfusate, placental perfusate cells, PINK cells, combined NK cells, or combinations thereof. In certain embodiments, elotuzumab and the human placental perfusate, placental perfusate cells, PINK cells, combined NK cells, or combinations thereof are administered via the same route of administration. In other embodiments, elotuzumab and the human placental perfusate, placental perfusate cells, PINK cells, combined NK cells, or combinations thereof are administered via different routes of administration. In certain embodiments, elotuzumab and the human placental perfusate, placental perfusate cells, PINK cells, combined NK cells, or combinations thereof are administered intravenously. In other embodiments, elotuzumab and the human placental perfusate, placental perfusate cells, PINK cells, combined NK cells, or combinations thereof are administered intracranially. In yet other embodiments, elotuzumab and the human placental perfusate, placental perfusate cells, PINK cells, combined NK cells, or combinations thereof are administered intrathecally. In still other embodiments, elotuzumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, elotuzumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and elotumumab is administered intracranially to an individual. In some embodiments, the human placental perfusate, human placental perfusate cells, PINK cells, combined NK cells, populations of cells comprising such cells, or combinations thereof, are administered intracranially, and elotumumab is administered intrathecally to an individual. In other embodiments, the human placental perfusate, human placental perfusate cells, PINK cells, combined NK cells, populations of cells comprising such cells, or combinations thereof, are administered intrathecally, and elotumumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and elotumumab is administered intrathecally to an individual. In some embodiments, the human placental perfusate, human placental perfusate cells, PINK cells, combined NK cells, populations of cells comprising such cells, or combinations thereof, are administered intracranially, and elotumumab is administered intravenously to an individual. In other embodiments, the human placental perfusate, human placental perfusate cells, PINK cells, combined NK cells, populations of cells comprising such cells, or combinations thereof, are administered intrathecally, and elotumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and elotumumab is administered by an implanted catheter to an individual. In some embodiments, the human placental perfusate, human placental perfusate cells, PINK cells, combined NK cells, populations of cells comprising such cells, or combinations thereof, are administered intracranially, and elotumumab is administered an implanted catheter to an individual. In other embodiments, the human placental perfusate, human placental perfusate cells, PINK cells, combined NK cells, populations of cells comprising such cells, or combinations thereof, are administered intrathecally, and elotumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and elotumumab is administered intrathecally to an individual. In some embodiments, the human placental perfusate, human placental perfusate cells, PINK cells, combined NK cells, populations of cells comprising such cells, or combinations thereof, are administered an implanted catheter, and elotumumab is administered intravenously to an individual. In other embodiments, the human placental perfusate, human placental perfusate cells, PINK cells, combined NK cells, populations of cells comprising such cells, or combinations thereof, are administered an implanted catheter, and elotumumab is administered intracranially to an individual.

In certain embodiments, the anti-CD20 antibody and the human placental perfusate, placental perfusate cells, PINK cells, combined NK cells, or combinations thereof are administered concurrently. In other embodiments, the anti-CD20 antibody is administered before administration of the human placental perfusate, placental perfusate cells, PINK cells, combined NK cells, or combinations thereof. In yet other embodiments, the anti-CD20 antibody is administered after administration of the human placental perfusate, placental perfusate cells, PINK cells, combined NK cells, or combinations thereof. In one embodiment, the anti-CD20 antibody is obinutuzumab. In another embodiment, the anti-CD20 antibody is ofatumumab. In yet another embodiment, the anti-CD20 antibody is rituximab. In certain embodiments, obinutuzumab and the human placental perfusate, placental perfusate cells, PINK cells, combined NK cells, or combinations thereof are administered concurrently. In other embodiments, obinutuzumab is administered before administration of the human placental perfusate, placental perfusate cells, PINK cells, combined NK cells, or combinations thereof. In yet other embodiments, obinutuzumab is administered after administration of the human placental perfusate, placental perfusate cells, PINK cells, combined NK cells, or combinations thereof. In certain embodiments, obinutuzumab and the human placental perfusate, placental perfusate cells, PINK cells, combined NK cells, or combinations thereof are administered via the same route of administration. In other embodiments, obinutuzumab and the human placental perfusate, placental perfusate cells, PINK cells, combined NK cells, or combinations thereof are administered via different routes of administration. In certain embodiments, obinutuzumab and the human placental perfusate, placental perfusate cells, PINK cells, combined NK cells, or combinations thereof are administered intravenously. In other embodiments, obinutuzumab and the human placental perfusate, placental perfusate cells, PINK cells, combined NK cells, or combinations thereof are administered intracranially. In yet other embodiments, obinutuzumab and the human placental perfusate, placental perfusate cells, PINK cells, combined NK cells, or combinations thereof are administered intrathecally. In still other embodiments, obinutuzumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, obinutuzumab and the human placental perfusate, placental perfusate cells, PINK cells, combined NK cells, or combinations thereof are delivered via intracranial or intravertebral needles and/or catheters with or without pump devices. In certain embodiments, ofatumumab and the human placental perfusate, placental perfusate cells, PINK cells, combined NK cells, or combinations thereof are administered concurrently. In other embodiments, ofatumumab is administered before administration of the human placental perfusate, placental perfusate cells, PINK cells, combined NK cells, or combinations thereof. In yet other embodiments, ofatumumab is administered after administration of the human placental perfusate, placental perfusate cells, PINK cells, combined NK cells, or combinations thereof. In certain embodiments, ofatumumab and the human placental perfusate, placental perfusate cells, PINK cells, combined NK cells, or combinations thereof are administered via the same route of administration. In other embodiments, ofatumumab and the human placental perfusate, placental perfusate cells, PINK cells, combined NK cells, or combinations thereof are administered via different routes of administration. In certain embodiments, ofatumumab and the human placental perfusate, placental perfusate cells, PINK cells, combined NK cells, or combinations thereof are administered intravenously. In other embodiments, ofatumumab and the human placental perfusate, placental perfusate cells, PINK cells, combined NK cells, or combinations thereof are administered intracranially. In yet other embodiments, ofatumumab and the human placental perfusate, placental perfusate cells, PINK cells, combined NK cells, or combinations thereof are administered intrathecally. In still other embodiments, ofatumumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, ofatumumab and the human placental perfusate, placental perfusate cells, PINK cells, combined NK cells, or combinations thereof are delivered via intracranial or intravertebral needles and/or catheters with or without pump devices. In certain embodiments, rituximab and the human placental perfusate, placental perfusate cells, PINK cells, combined NK cells, or combinations thereof are administered concurrently. In other embodiments, rituximab is administered before administration of the human placental perfusate, placental perfusate cells, PINK cells, combined NK cells, or combinations thereof. In yet other embodiments, rituximab is administered after administration of the human placental perfusate, placental perfusate cells, PINK cells, combined NK cells, or combinations thereof. In certain embodiments, rituximab and the human placental perfusate, placental perfusate cells, PINK cells, combined NK cells, or combinations thereof are administered via the same route of administration. In other embodiments, rituximab and the human placental perfusate, placental perfusate cells, PINK cells, combined NK cells, or combinations thereof are administered via different routes of administration. In certain embodiments, rituximab and the human placental perfusate, placental perfusate cells, PINK cells, combined NK cells, or combinations thereof are administered intravenously. In other embodiments, rituximab and the human placental perfusate, placental perfusate cells, PINK cells, combined NK cells, or combinations thereof are administered intracranially. In yet other embodiments, rituximab and the human placental perfusate, placental perfusate cells, PINK cells, combined NK cells, or combinations thereof are administered intrathecally. In still other embodiments, rituximab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, rituximab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and obinutumumab is administered intracranially to an individual. In some embodiments, the human placental perfusate, human placental perfusate cells, PINK cells, combined NK cells, populations of cells comprising such cells, or combinations thereof, are administered intracranially, and obinutumumab is administered intrathecally to an individual. In other embodiments, the human placental perfusate, human placental perfusate cells, PINK cells, combined NK cells, populations of cells comprising such cells, or combinations thereof, are administered intrathecally, and obinutumumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and obinutumumab is administered intrathecally to an individual. In some embodiments, the human placental perfusate, human placental perfusate cells, PINK cells, combined NK cells, populations of cells comprising such cells, or combinations thereof, are administered intracranially, and obinutumumab is administered intravenously to an individual. In other embodiments, the human placental perfusate, human placental perfusate cells, PINK cells, combined NK cells, populations of cells comprising such cells, or combinations thereof, are administered intrathecally, and obinutumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and obinutumumab is administered by an implanted catheter to an individual. In some embodiments, the human placental perfusate, human placental perfusate cells, PINK cells, combined NK cells, populations of cells comprising such cells, or combinations thereof, are administered intracranially, and obinutumumab is administered an implanted catheter to an individual. In other embodiments, the human placental perfusate, human placental perfusate cells, PINK cells, combined NK cells, populations of cells comprising such cells, or combinations thereof, are administered intrathecally, and obinutumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and obinutumumab is administered intrathecally to an individual. In some embodiments, the human placental perfusate, human placental perfusate cells, PINK cells, combined NK cells, populations of cells comprising such cells, or combinations thereof, are administered an implanted catheter, and obinutumumab is administered intravenously to an individual. In other embodiments, the human placental perfusate, human placental perfusate cells, PINK cells, combined NK cells, populations of cells comprising such cells, or combinations thereof, are administered an implanted catheter, and obinutumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and ofatumumab is administered intracranially to an individual. In some embodiments, the human placental perfusate, human placental perfusate cells, PINK cells, combined NK cells, populations of cells comprising such cells, or combinations thereof, are administered intracranially, and ofatumumab is administered intrathecally to an individual. In other embodiments, the human placental perfusate, human placental perfusate cells, PINK cells, combined NK cells, populations of cells comprising such cells, or combinations thereof, are administered intrathecally, and ofatumumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and ofatumumab is administered intrathecally to an individual. In some embodiments, the human placental perfusate, human placental perfusate cells, PINK cells, combined NK cells, populations of cells comprising such cells, or combinations thereof, are administered intracranially, and ofatumumab is administered intravenously to an individual. In other embodiments, the human placental perfusate, human placental perfusate cells, PINK cells, combined NK cells, populations of cells comprising such cells, or combinations thereof, are administered intrathecally, and ofatumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and ofatumumab is administered by an implanted catheter to an individual. In some embodiments, the human placental perfusate, human placental perfusate cells, PINK cells, combined NK cells, populations of cells comprising such cells, or combinations thereof, are administered intracranially, and ofatumumab is administered an implanted catheter to an individual. In other embodiments, the human placental perfusate, human placental perfusate cells, PINK cells, combined NK cells, populations of cells comprising such cells, or combinations thereof, are administered intrathecally, and ofatumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and ofatumumab is administered intrathecally to an individual. In some embodiments, the human placental perfusate, human placental perfusate cells, PINK cells, combined NK cells, populations of cells comprising such cells, or combinations thereof, are administered an implanted catheter, and ofatumumab is administered intravenously to an individual. In other embodiments, the human placental perfusate, human placental perfusate cells, PINK cells, combined NK cells, populations of cells comprising such cells, or combinations thereof, are administered an implanted catheter, and ofatumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and rituximab is administered intracranially to an individual. In some embodiments, the human placental perfusate, human placental perfusate cells, PINK cells, combined NK cells, populations of cells comprising such cells, or combinations thereof, are administered intracranially, and rituximab is administered intrathecally to an individual. In other embodiments, the human placental perfusate, human placental perfusate cells, PINK cells, combined NK cells, populations of cells comprising such cells, or combinations thereof, are administered intrathecally, and rituximab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and rituximab is administered intrathecally to an individual. In some embodiments, the human placental perfusate, human placental perfusate cells, PINK cells, combined NK cells, populations of cells comprising such cells, or combinations thereof, are administered intracranially, and rituximab is administered intravenously to an individual. In other embodiments, the human placental perfusate, human placental perfusate cells, PINK cells, combined NK cells, populations of cells comprising such cells, or combinations thereof, are administered intrathecally, and rituximab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and rituximab is administered by an implanted catheter to an individual. In some embodiments, the human placental perfusate, human placental perfusate cells, PINK cells, combined NK cells, populations of cells comprising such cells, or combinations thereof, are administered intracranially, and rituximab is administered an implanted catheter to an individual. In other embodiments, the human placental perfusate, human placental perfusate cells, PINK cells, combined NK cells, populations of cells comprising such cells, or combinations thereof, are administered intrathecally, and rituximab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and rituximab is administered intrathecally to an individual. In some embodiments, the human placental perfusate, human placental perfusate cells, PINK cells, combined NK cells, populations of cells comprising such cells, or combinations thereof, are administered an implanted catheter, and rituximab is administered intravenously to an individual. In other embodiments, the human placental perfusate, human placental perfusate cells, PINK cells, combined NK cells, populations of cells comprising such cells, or combinations thereof, are administered an implanted catheter, and rituximab is administered intracranially to an individual.

In certain embodiments, the anti-GD2 antibody and the human placental perfusate, placental perfusate cells, PINK cells, combined NK cells, or combinations thereof are administered concurrently. In other embodiments, the anti-GD2 antibody is administered before administration of the human placental perfusate, placental perfusate cells, PINK cells, combined NK cells, or combinations thereof. In yet other embodiments, the anti-GD2 antibody is administered after administration of the human placental perfusate, placental perfusate cells, PINK cells, combined NK cells, or combinations thereof. In one embodiment, the anti-GD2 antibody is dinutuximab. In certain embodiments, dinutuximab and the human placental perfusate, placental perfusate cells, PINK cells, combined NK cells, or combinations thereof are administered concurrently. In other embodiments, dinutuximab is administered before administration of the human placental perfusate, placental perfusate cells, PINK cells, combined NK cells, or combinations thereof. In yet other embodiments, dinutuximab is administered after administration of the human placental perfusate, placental perfusate cells, PINK cells, combined NK cells, or combinations thereof. In certain embodiments, dinutuximab and the human placental perfusate, placental perfusate cells, PINK cells, combined NK cells, or combinations thereof are administered via the same route of administration. In other embodiments, dinutuximab and the human placental perfusate, placental perfusate cells, PINK cells, combined NK cells, or combinations thereof are administered via different routes of administration. In certain embodiments, dinutuximab and the human placental perfusate, placental perfusate cells, PINK cells, combined NK cells, or combinations thereof are administered intravenously. In other embodiments, dinutuximab and the human placental perfusate, placental perfusate cells, PINK cells, combined NK cells, or combinations thereof are administered intracranially. In yet other embodiments, dinutuximab and the human placental perfusate, placental perfusate cells, PINK cells, combined NK cells, or combinations thereof are administered intrathecally. In still other embodiments, dinutuximab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, dinutuximab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and dinutuximab is administered intracranially to an individual. In some embodiments, the human placental perfusate, human placental perfusate cells, PINK cells, combined NK cells, populations of cells comprising such cells, or combinations thereof, are administered intracranially, and dinutuximab is administered intrathecally to an individual. In other embodiments, the human placental perfusate, human placental perfusate cells, PINK cells, combined NK cells, populations of cells comprising such cells, or combinations thereof, are administered intrathecally, and dinutuximab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and dinutuximab is administered intrathecally to an individual. In some embodiments, the human placental perfusate, human placental perfusate cells, PINK cells, combined NK cells, populations of cells comprising such cells, or combinations thereof, are administered intracranially, and dinutuximab is administered intravenously to an individual. In other embodiments, the human placental perfusate, human placental perfusate cells, PINK cells, combined NK cells, populations of cells comprising such cells, or combinations thereof, are administered intrathecally, and dinutuximab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and dinutuximab is administered by an implanted catheter to an individual. In some embodiments, the human placental perfusate, human placental perfusate cells, PINK cells, combined NK cells, populations of cells comprising such cells, or combinations thereof, are administered intracranially, and dinutuximab is administered an implanted catheter to an individual. In other embodiments, the human placental perfusate, human placental perfusate cells, PINK cells, combined NK cells, populations of cells comprising such cells, or combinations thereof, are administered intrathecally, and dinutuximab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and dinutuximab is administered intrathecally to an individual. In some embodiments, the human placental perfusate, human placental perfusate cells, PINK cells, combined NK cells, populations of cells comprising such cells, or combinations thereof, are administered an implanted catheter, and dinutuximab is administered intravenously to an individual. In other embodiments, the human placental perfusate, human placental perfusate cells, PINK cells, combined NK cells, populations of cells comprising such cells, or combinations thereof, are administered an implanted catheter, and dinutuximab is administered intracranially to an individual.

In certain embodiments, the anti-HER2 antibody and the human placental perfusate, placental perfusate cells, PINK cells, combined NK cells, or combinations thereof are administered concurrently. In other embodiments, the anti-HER2 antibody is administered before administration of the human placental perfusate, placental perfusate cells, PINK cells, combined NK cells, or combinations thereof. In yet other embodiments, the anti-HER2 antibody is administered after administration of the human placental perfusate, placental perfusate cells, PINK cells, combined NK cells, or combinations thereof. In one embodiment, the anti-HER2 antibody is trastuzumab. In certain embodiments, trastuzumab and the human placental perfusate, placental perfusate cells, PINK cells, combined NK cells, or combinations thereof are administered concurrently. In other embodiments, trastuzumab is administered before administration of the human placental perfusate, placental perfusate cells, PINK cells, combined NK cells, or combinations thereof. In yet other embodiments, trastuzumab is administered after administration of the human placental perfusate, placental perfusate cells, PINK cells, combined NK cells, or combinations thereof. In certain embodiments, trastuzumab and the human placental perfusate, placental perfusate cells, PINK cells, combined NK cells, or combinations thereof are administered via the same route of administration. In other embodiments, trastuzumab and the human placental perfusate, placental perfusate cells, PINK cells, combined NK cells, or combinations thereof are administered via different routes of administration. In certain embodiments, trastuzumab and the human placental perfusate, placental perfusate cells, PINK cells, combined NK cells, or combinations thereof are administered intravenously. In other embodiments, trastuzumab and the human placental perfusate, placental perfusate cells, PINK cells, combined NK cells, or combinations thereof are administered intracranially. In yet other embodiments, trastuzumab and the human placental perfusate, placental perfusate cells, PINK cells, combined NK cells, or combinations thereof are administered intrathecally. In still other embodiments, trastuzumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, trastuzumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and trastuzumab is administered intracranially to an individual. In some embodiments, the human placental perfusate, human placental perfusate cells, PINK cells, combined NK cells, populations of cells comprising such cells, or combinations thereof, are administered intracranially, and trastuzumab is administered intrathecally to an individual. In other embodiments, the human placental perfusate, human placental perfusate cells, PINK cells, combined NK cells, populations of cells comprising such cells, or combinations thereof, are administered intrathecally, and trastuzumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and trastuzumab is administered intrathecally to an individual. In some embodiments, the human placental perfusate, human placental perfusate cells, PINK cells, combined NK cells, populations of cells comprising such cells, or combinations thereof, are administered intracranially, and trastuzumab is administered intravenously to an individual. In other embodiments, the human placental perfusate, human placental perfusate cells, PINK cells, combined NK cells, populations of cells comprising such cells, or combinations thereof, are administered intrathecally, and trastuzumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and trastuzumab is administered by an implanted catheter to an individual. In some embodiments, the human placental perfusate, human placental perfusate cells, PINK cells, combined NK cells, populations of cells comprising such cells, or combinations thereof, are administered intracranially, and trastuzumab is administered an implanted catheter to an individual. In other embodiments, the human placental perfusate, human placental perfusate cells, PINK cells, combined NK cells, populations of cells comprising such cells, or combinations thereof, are administered intrathecally, and trastuzumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and trastuzumab is administered intrathecally to an individual. In some embodiments, the human placental perfusate, human placental perfusate cells, PINK cells, combined NK cells, populations of cells comprising such cells, or combinations thereof, are administered an implanted catheter, and trastuzumab is administered intravenously to an individual. In other embodiments, the human placental perfusate, human placental perfusate cells, PINK cells, combined NK cells, populations of cells comprising such cells, or combinations thereof, are administered an implanted catheter, and trastuzumab is administered intracranially to an individual.

In certain embodiments, the anti-PD-L1 antibody and the human placental perfusate, placental perfusate cells, PINK cells, combined NK cells, or combinations thereof are administered concurrently. In other embodiments, the anti-PD-L1 antibody is administered before administration of the human placental perfusate, placental perfusate cells, PINK cells, combined NK cells, or combinations thereof. In yet other embodiments, the anti-PD-L1 antibody is administered after administration of the human placental perfusate, placental perfusate cells, PINK cells, combined NK cells, or combinations thereof. In one embodiment, the anti-PD-L1 antibody is atezolizumab. In another embodiment, the anti-PD-L1 antibody is avelumab. In certain embodiments, atezolizumab and the human placental perfusate, placental perfusate cells, PINK cells, combined NK cells, or combinations thereof are administered concurrently. In other embodiments, atezolizumab is administered before administration of the human placental perfusate, placental perfusate cells, PINK cells, combined NK cells, or combinations thereof. In yet other embodiments, atezolizumab is administered after administration of the human placental perfusate, placental perfusate cells, PINK cells, combined NK cells, or combinations thereof. In certain embodiments, atezolizumab and the human placental perfusate, placental perfusate cells, PINK cells, combined NK cells, or combinations thereof are administered via the same route of administration. In other embodiments, atezolizumab and the human placental perfusate, placental perfusate cells, PINK cells, combined NK cells, or combinations thereof are administered via different routes of administration. In certain embodiments, atezolizumab and the human placental perfusate, placental perfusate cells, PINK cells, combined NK cells, or combinations thereof are administered intravenously. In other embodiments, atezolizumab and the human placental perfusate, placental perfusate cells, PINK cells, combined NK cells, or combinations thereof are administered intracranially. In yet other embodiments, atezolizumab and the human placental perfusate, placental perfusate cells, PINK cells, combined NK cells, or combinations thereof are administered intrathecally. In still other embodiments, atezolizumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, atezolizumab and the human placental perfusate, placental perfusate cells, PINK cells, combined NK cells, or combinations thereof are delivered via intracranial or intravertebral needles and/or catheters with or without pump devices. In certain embodiments, avelumab and the human placental perfusate, placental perfusate cells, PINK cells, combined NK cells, or combinations thereof are administered concurrently. In other embodiments, avelumab is administered before administration of the human placental perfusate, placental perfusate cells, PINK cells, combined NK cells, or combinations thereof. In yet other embodiments, avelumab is administered after administration of the human placental perfusate, placental perfusate cells, PINK cells, combined NK cells, or combinations thereof. In certain embodiments, avelumab and the human placental perfusate, placental perfusate cells, PINK cells, combined NK cells, or combinations thereof are administered via the same route of administration. In other embodiments, avelumab and the human placental perfusate, placental perfusate cells, PINK cells, combined NK cells, or combinations thereof are administered via different routes of administration. In certain embodiments, avelumab and the human placental perfusate, placental perfusate cells, PINK cells, combined NK cells, or combinations thereof are administered intravenously. In other embodiments, avelumab and the human placental perfusate, placental perfusate cells, PINK cells, combined NK cells, or combinations thereof are administered intracranially. In yet other embodiments, avelumab and the human placental perfusate, placental perfusate cells, PINK cells, combined NK cells, or combinations thereof are administered intrathecally. In still other embodiments, avelumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, avelumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and atezolizumab is administered intracranially to an individual. In some embodiments, the human placental perfusate, human placental perfusate cells, PINK cells, combined NK cells, populations of cells comprising such cells, or combinations thereof, are administered intracranially, and atezolizumab is administered intrathecally to an individual. In other embodiments, the human placental perfusate, human placental perfusate cells, PINK cells, combined NK cells, populations of cells comprising such cells, or combinations thereof, are administered intrathecally, and atezolizumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and atezolizumab is administered intrathecally to an individual. In some embodiments, the human placental perfusate, human placental perfusate cells, PINK cells, combined NK cells, populations of cells comprising such cells, or combinations thereof, are administered intracranially, and atezolizumab is administered intravenously to an individual. In other embodiments, the human placental perfusate, human placental perfusate cells, PINK cells, combined NK cells, populations of cells comprising such cells, or combinations thereof, are administered intrathecally, and atezolizumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and atezolizumab is administered by an implanted catheter to an individual. In some embodiments, the human placental perfusate, human placental perfusate cells, PINK cells, combined NK cells, populations of cells comprising such cells, or combinations thereof, are administered intracranially, and atezolizumab is administered an implanted catheter to an individual. In other embodiments, the human placental perfusate, human placental perfusate cells, PINK cells, combined NK cells, populations of cells comprising such cells, or combinations thereof, are administered intrathecally, and atezolizumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and atezolizumab is administered intrathecally to an individual. In some embodiments, the human placental perfusate, human placental perfusate cells, PINK cells, combined NK cells, populations of cells comprising such cells, or combinations thereof, are administered an implanted catheter, and atezolizumab is administered intravenously to an individual. In other embodiments, the human placental perfusate, human placental perfusate cells, PINK cells, combined NK cells, populations of cells comprising such cells, or combinations thereof, are administered an implanted catheter, and atezolizumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and avelumab is administered intracranially to an individual. In some embodiments, the human placental perfusate, human placental perfusate cells, PINK cells, combined NK cells, populations of cells comprising such cells, or combinations thereof, are administered intracranially, and avelumab is administered intrathecally to an individual. In other embodiments, the human placental perfusate, human placental perfusate cells, PINK cells, combined NK cells, populations of cells comprising such cells, or combinations thereof, are administered intrathecally, and avelumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and avelumab is administered intrathecally to an individual. In some embodiments, the human placental perfusate, human placental perfusate cells, PINK cells, combined NK cells, populations of cells comprising such cells, or combinations thereof, are administered intracranially, and avelumab is administered intravenously to an individual. In other embodiments, the human placental perfusate, human placental perfusate cells, PINK cells, combined NK cells, populations of cells comprising such cells, or combinations thereof, are administered intrathecally, and avelumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and avelumab is administered by an implanted catheter to an individual. In some embodiments, the human placental perfusate, human placental perfusate cells, PINK cells, combined NK cells, populations of cells comprising such cells, or combinations thereof, are administered intracranially, and avelumab is administered an implanted catheter to an individual. In other embodiments, the human placental perfusate, human placental perfusate cells, PINK cells, combined NK cells, populations of cells comprising such cells, or combinations thereof, are administered intrathecally, and avelumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and avelumab is administered intrathecally to an individual. In some embodiments, the human placental perfusate, human placental perfusate cells, PINK cells, combined NK cells, populations of cells comprising such cells, or combinations thereof, are administered an implanted catheter, and avelumab is administered intravenously to an individual. In other embodiments, the human placental perfusate, human placental perfusate cells, PINK cells, combined NK cells, populations of cells comprising such cells, or combinations thereof, are administered an implanted catheter, and avelumab is administered intracranially to an individual.

In certain embodiments, the anti-EGFR antibody and the human placental perfusate, placental perfusate cells, PINK cells, combined NK cells, or combinations thereof are administered concurrently. In other embodiments, the anti-EGFR antibody is administered before administration of the human placental perfusate, placental perfusate cells, PINK cells, combined NK cells, or combinations thereof. In yet other embodiments, the anti-EGFR antibody is administered after administration of the human placental perfusate, placental perfusate cells, PINK cells, combined NK cells, or combinations thereof. In one embodiment, the anti-EGFR antibody is cetuximab. In certain embodiments, cetuximab and the human placental perfusate, placental perfusate cells, PINK cells, combined NK cells, or combinations thereof are administered concurrently. In other embodiments, cetuximab is administered before administration of the human placental perfusate, placental perfusate cells, PINK cells, combined NK cells, or combinations thereof. In yet other embodiments, cetuximab is administered after administration of the human placental perfusate, placental perfusate cells, PINK cells, combined NK cells, or combinations thereof. In certain embodiments, cetuximab and the human placental perfusate, placental perfusate cells, PINK cells, combined NK cells, or combinations thereof are administered via the same route of administration. In other embodiments, cetuximab and the human placental perfusate, placental perfusate cells, PINK cells, combined NK cells, or combinations thereof are administered via different routes of administration. In certain embodiments, cetuximab and the human placental perfusate, placental perfusate cells, PINK cells, combined NK cells, or combinations thereof are administered intravenously. In other embodiments, cetuximab and the human placental perfusate, placental perfusate cells, PINK cells, combined NK cells, or combinations thereof are administered intracranially. In yet other embodiments, cetuximab and the human placental perfusate, placental perfusate cells, PINK cells, combined NK cells, or combinations thereof are administered intrathecally. In still other embodiments, cetuximab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, cetuximab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and cetuximab is administered intracranially to an individual. In some embodiments, the human placental perfusate, human placental perfusate cells, PINK cells, combined NK cells, populations of cells comprising such cells, or combinations thereof, are administered intracranially, and cetuximab is administered intrathecally to an individual. In other embodiments, the human placental perfusate, human placental perfusate cells, PINK cells, combined NK cells, populations of cells comprising such cells, or combinations thereof, are administered intrathecally, and cetuximab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and cetuximab is administered intrathecally to an individual. In some embodiments, the human placental perfusate, human placental perfusate cells, PINK cells, combined NK cells, populations of cells comprising such cells, or combinations thereof, are administered intracranially, and cetuximab is administered intravenously to an individual. In other embodiments, the human placental perfusate, human placental perfusate cells, PINK cells, combined NK cells, populations of cells comprising such cells, or combinations thereof, are administered intrathecally, and cetuximab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and cetuximab is administered by an implanted catheter to an individual. In some embodiments, the human placental perfusate, human placental perfusate cells, PINK cells, combined NK cells, populations of cells comprising such cells, or combinations thereof, are administered intracranially, and cetuximab is administered an implanted catheter to an individual. In other embodiments, the human placental perfusate, human placental perfusate cells, PINK cells, combined NK cells, populations of cells comprising such cells, or combinations thereof, are administered intrathecally, and cetuximab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and cetuximab is administered intrathecally to an individual. In some embodiments, the human placental perfusate, human placental perfusate cells, PINK cells, combined NK cells, populations of cells comprising such cells, or combinations thereof, are administered an implanted catheter, and cetuximab is administered intravenously to an individual. In other embodiments, the human placental perfusate, human placental perfusate cells, PINK cells, combined NK cells, populations of cells comprising such cells, or combinations thereof, are administered an implanted catheter, and cetuximab is administered intracranially to an individual.

The human placental perfusate, human placental perfusate cells, PINK cells, combined NK cells, populations of cells comprising such cells, or combinations thereof, and the antibody can be administered to an individual in a composition, e.g., a matrix, hydrogel, scaffold, or the like that comprise the cells and the antibody.

In one embodiment, the cells provided herein are seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which placental stem cells can be seeded are described in Hariri, U.S. Application Publication No. 2004/0048796, the disclosure of which is hereby incorporated by reference in its entirety.

In another embodiment, the human placental perfusate, human placental perfusate cells, PINK cells, combined NK cells, populations of cells comprising such cells, or combinations thereof, and the antibody are suspended in a hydrogel solution suitable for, e.g., injection. Suitable hydrogels for such compositions include self-assembling peptides, such as RAD16. In one embodiment, a hydrogel solution comprising the cells and the antibody can be allowed to harden, for instance in a mold, to form a matrix having cells and antibody dispersed therein for implantation. The cells in such a matrix can also be cultured so that the cells are mitotically expanded prior to implantation. The hydrogel can be, for example, an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix of the invention is biodegradable.

In some embodiments of the invention, the formulation comprises an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

The human placental perfusate cells, PINK cells, combined NK cells, populations of cells comprising such cells, or combinations thereof, can be seeded onto a three-dimensional framework or scaffold and implanted in vivo. Such a framework can be implanted in combination with any one or more growth factors, cells, drugs or other components that stimulate tissue formation or otherwise enhance or improve the practice of the invention.

Examples of scaffolds that can be used in the present invention include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

The human placental perfusate cells, PINK cells, combined NK cells, populations of cells comprising such cells, or combinations thereof, can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, the human placental perfusate cells, PINK cells, combined NK cells, populations of cells comprising such cells, or combinations thereof, can be seeded onto, or contacted with, a felt, which can be, e.g., composed of a multifilament yarn made from a bioabsorbable material such as PGA, PLA, PCL copolymers or blends, or hyaluronic acid.

The human placental perfusate cells, PINK cells, combined NK cells, populations of cells comprising such cells, or combinations thereof, can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1 M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with placental stem cells.

### 5.10. Compositions Comprising Human Placental Perfusate, Placental Perfusate Cells, PINK Cells, Combined NK Cells, or Combinations Thereof in Combination with an Antibody and Methods of Use Thereof

In another aspect, provided herein are compositions comprising human placental perfusate, placental perfusate cells, PINK cells, combined NK cells, or combinations thereof, in combination with an antibody. Any combinations of human placental perfusate, placental perfusate cells, PINK cells, combined NK cells, or combinations thereof, and antibodies described herein are contemplated. In certain embodiments, the composition comprises more than one antibody. In some embodiments, the composition comprises two, three, four, or five antibodies. In other embodiments, the composition comprises human placental perfusate, placental perfusate cells, PINK cells, combined NK cells, or combinations thereof, and an antibody, and further comprises one or more additional agents. In yet other embodiments, the additional agents are anti-cancer agents selected from the list described in section 5.7. In still other embodiments, the additional agents are excipients, buffers, stabilizers, media, media supplements, antibiotics, or any additives known in the art that help maintain the stability and/or activity of the compositions.

In another aspect, provided herein are methods of suppressing growth or proliferation of tumor cells by contacting the tumor cells with the composition that comprises human placental perfusate, placental perfusate cells, PINK cells, combined NK cells, or combinations thereof, in combination with an antibody, as described herein. Also provided herein are methods of treating an individual having tumor cells by administering a therapeutically effective amount of the composition that comprises human placental perfusate, placental perfusate cells, PINK cells, combined NK cells, or combinations thereof, in combination with an antibody, as described herein, to the individual.

In a specific embodiment, the tumor cells are blood cancer cells. In another embodiment, the tumor cells are solid tumor cells. In various specific embodiments, the tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells. In one embodiment, the tumor cells are glioblastoma cells. In one embodiment, the tumor cells are neuroblastoma cells. In one embodiment, the tumor cells are osteosarcoma cells. In one embodiment, the tumor cells are melanoma cells. In one embodiment, the tumor cells are ovarian cancer cells. In one embodiment, the tumor cells are primary ductal carcinoma cells. In one embodiment, the tumor cells are leukemia cells. In one embodiment, the tumor cells are acute T cell leukemia cells. In one embodiment, the tumor cells are AML cells. In one embodiment, the tumor cells are CML cells. In one embodiment, the tumor cells are ALL cells. In one embodiment, the tumor cells are CLL cells. In one embodiment, the tumor cells are NHL cells. In one embodiment, the tumor cells are breast cancer cells. In one embodiment, the tumor cells are bladder cancer cells. In one embodiment, the tumor cells are Merkel cell carcinoma cells. In one embodiment, the tumor cells are head and neck cancer cells. In one embodiment, the tumor cells are lung carcinoma cells. In one embodiment, the tumor cells are colon adenocarcinoma cells. In one embodiment, the tumor cells are histiocytic lymphoma cells. In one embodiment, the tumor cells are multiple myeloma cells. In one embodiment, the tumor cells are retinoblastoma cells. In one embodiment, the tumor cells are colorectal carcinoma cells. In one embodiment, the tumor cells are colorectal adenocarcinoma cells.

### 6. EXAMPLES

### 6.1. Example 1: Preparation of PNK Cells

PINK cells, PNK, were generated using the two-step expansion and differentiation method described in U.S. Pat. No. 8,926,964 then frozen. Prior to the cytotoxicity experiments below, PNK cells were thawed, recovered with medium for 3 days, and resuspended at 1×10⁶ cells/mL with assay medium (RPMI 1640 medium supplemented with 10% fetal bovine serum (FBS) and antibiotics (1% penicillin and streptomycin)) for cytotoxicity assay.

### 6.2. Example 2: Expression of GD2 on Different Solid Tumor Cell Lines

A panel of solid tumor cell lines (Table 1) was evaluated for expression of GD2. 1×105 cells from each solid tumor cell line were stained with PE anti-human GD2 (BioLegend, San Diego, Calif., Cat #357304) at 40° C. for 15 min. Unstained tumor cells and tumor cells stained with PE mouse IgG2a, K isotype control (BioLegend, Cat #400214) were used as controls.

TABLE 1
Solid Tumor Cell Lines Evaluated for Expression of GD2

| Tumor Cell Line | Tumor Type |
|---|---|
| Caov-3 | Ovarian cancer |
| HT-144 | Melanoma |
| M-21 | Melanoma |
| NMB-7 | Neuroblastoma |
| SK-MES-1 | Lung cancer |
| U-251 | Glioblastoma |
| U-87MG | Glioblastoma |

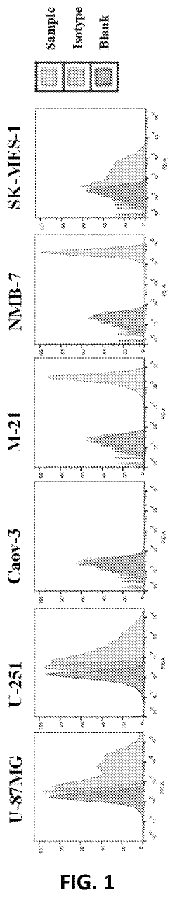

FIG. 1 shows GD-2 expression in exemplary solid tumor cell lines. The expression levels of GD-2 in different solid tumor cell lines are summarized in Table 2 below.

TABLE 2
Expression Level of GD2 in Exemplary Solid Tumor Cell Lines

| | U-87MG | U-251 | Caov-3 | M-21 | NMB-7 | SK-MES-1 |
|---|---|---|---|---|---|---|
| GD2 | +++ | +++ | − | +++++ | +++++ | +++ |
| Expres- | | | | | | |
| sion | | | | | | |

### 6.3. Example 3: In Vitro Anti-Tumor Activity of PNK Cells in Combination with Anti-GD2 Antibody Unituxin® (Dinutuximab)

1×10⁷ cells from each solid tumor cell line were first labeled with 7.5 μM PKH26 fluorescent dye (Sigma-Aldrich, St Louis, Mo., Cat # PKH26-GL), then treated with Unituxin® (dinutuximab, from United Therapeutics, Silver Spring, Md.) at different concentration of 0, 0.01, 0.1, 1, 3, 10, 30, or 100 μg/mL at 37° C. for 30 min. Human IgG1 (BioLegend, Cat #403502) at corresponding concentrations was used as isotype control. The treated cells were washed twice with assay medium (see section 5.1). Four-hour cytotoxicity assay was performed by using PNK cells as effector cells and PKH26 labeled, Unituxin® or human IgG1 treated tumor cells as target cells. Target cell number was fixed at 1×10⁴, while the number of PNK cells was determined based on different effector to target (E:T) ratios, such as 10:1, 3:1, and 1:1. Target cells were incubated with PNK cells in 96-well flat-bottom tissue culture plates in 200 μL of the assay medium for 4 hours at 37° C. in 5% CO₂. After incubation, cells were harvested and TO-PRO-3 (Invitrogen, Carlsbad, Calif., Cat # T3605), a membrane-impermeable DNA stain, was added to the cultures at 1 μM final concentration to identify dead cells (TO-PRO-3+). To determine spontaneous target cell death, PKH26-labeled target cells were cultured alone for the duration of the assay. As a positive control for dead cells, 1×10⁵ labeled target cells were permeabilized with 300 μL of Cytofix/Cytoperm buffer (BD Biosciences, San Jose, Calif., Cat #554722) for 20 minutes at 4° C. Data were acquired on FACSCanto II (BD Biosciences) and analyzed using FlowJo software (Tree Star, Ashland, Oreg.).

The percentage of dead target cells in each sample was calculated as follows: % TO-PRO-3⁺PKH26⁺ cells/(% TO-PRO-3⁺PKH26⁺+% TO-PRO-3⁻PKH26⁺)*100%. The percentage of cytotoxicity was calculated by subtracting the percentage of dead target cells in cultures of the target cells alone from the percentage of dead target cells in co-cultures of PNK cells and the target cells. Results from different experiments were reported as mean±standard deviation of the mean. Two-way analysis of variance was used to assess if there is any interaction between PNK cells and Unituxin®.

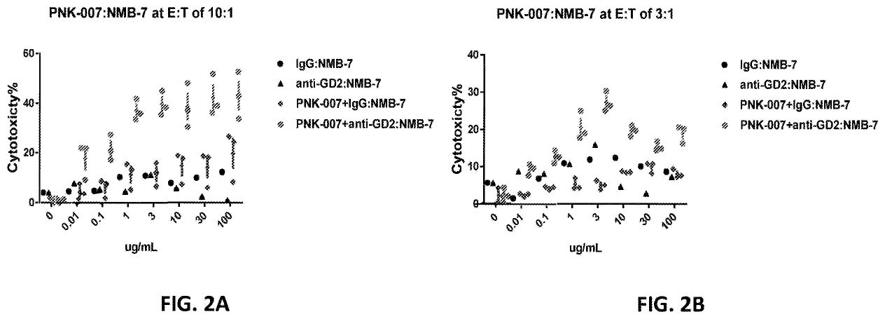

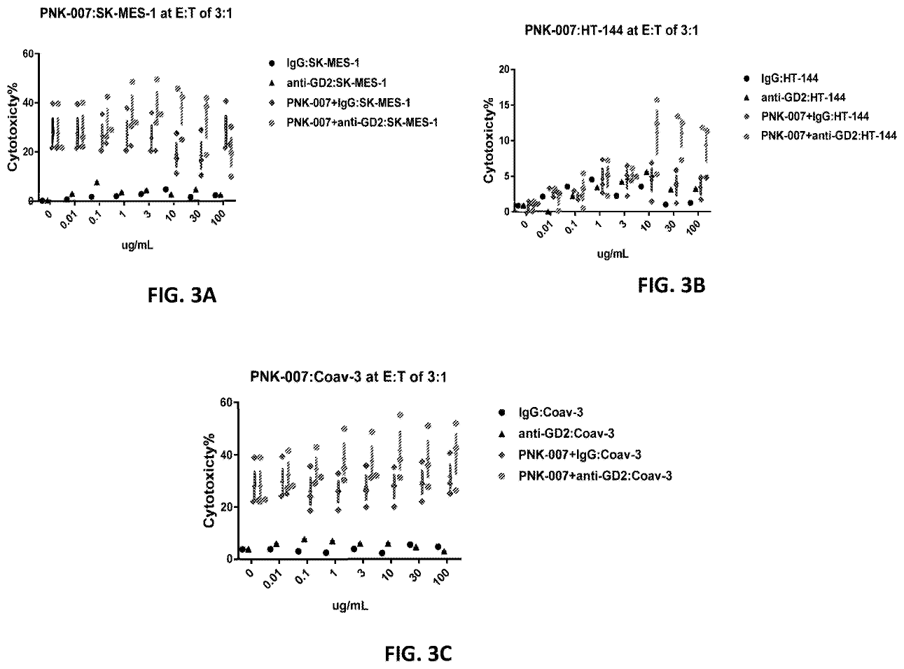

FIGS. 2A and 2B showed that the cytotoxicity of PNK cells against neuroblastoma cell line NMB-7 increased significantly in presence of Unituxin®, compared with that in presence of IgG1 control, at E:T ratio of 10:1 (FIG. 2A) or 3:1 (FIG. 2B). The cytotoxicity of PNK cells against NMB-7 cells was Unituxin® concentration-dependent.

Similarly, the cytotoxicity of PNK cells against human lung cancer cell line SK-MES-1 (FIG. 3A), human melanoma cell line HT-144 (FIG. 3B), or human ovarian cancer cell line Coav-3 (FIG. 3C), increased significantly in presence of Unituxin®, compared with that in presence of IgG1 control, at E:T ratio of 3:1.

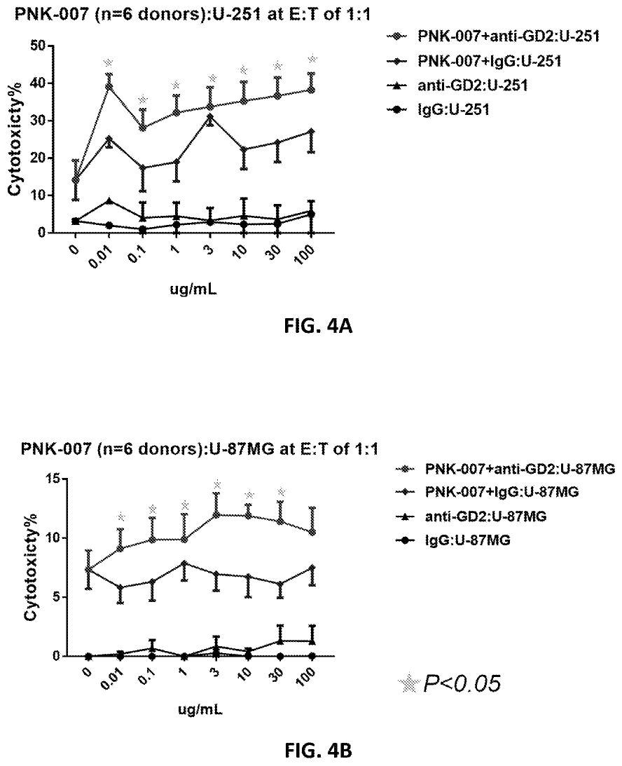

In addition, the cytotoxicity of PNK cells against human glioblastoma cell line U-251 significantly increased in presence of Unituxin®, compared with that in presence of IgG1 control, at E:T ratio of 1:1 (FIG. 4A). Similarly, the cytotoxicity of PNK cells against another human glioblastoma cell line U-87MG also was increased significantly in presence of Unituxin®, compared with that in presence of IgG1 control, at E:T ratio of 1:1 (FIG. 4B).

### 6.4. Example 4: Expression of CD38 on Different Human Lymphoma Tumor Cell Lines

Expression of CD38 on three different human lymphoma tumor cell lines-Daudi, Raji, and HS-sultan—was studied. 1×10⁵ cells from each human lymphoma tumor cell line were stained with FITC mouse anti-human CD38 (BD Bioscience, Cat #560982) at 4° C. for 15 min. Unstained tumor cells and tumor cells stained with FITC mouse IgG1, κ isotype control (BD Bioscience, Cat #555748) were used as controls.

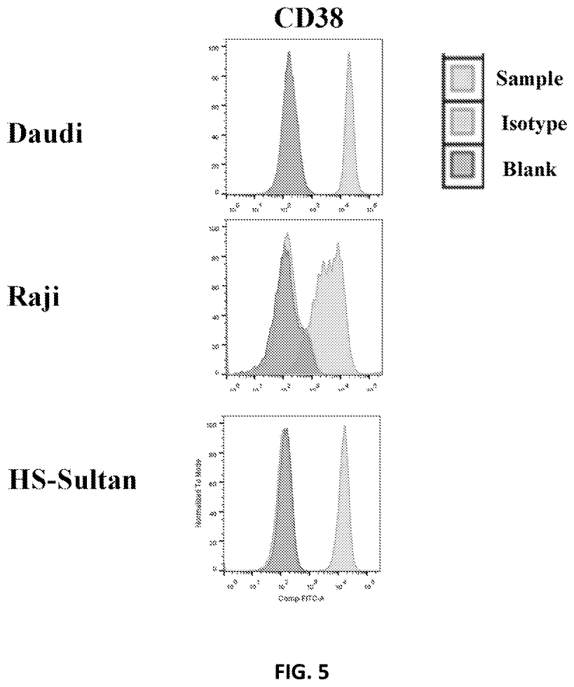

FIG. 5 shows that all three human lymphoma cell lines express CD38. The percentage of CD38 expression in Daudi cells and HS-sultan cells was comparable and higher than that in Raji cells.

### 6.5. Example 5: In Vitro Anti-Tumor Activity of PNK Cells in Combination with Anti-CD38 Antibody Daratumumab

1×10⁷ cells from each lymphoma cell line, Daudi, HS-sultan, and Raji, were first labeled with 7.5 μM PKH26 fluorescent dye, then treated with daratumumab (customized by Creative Biolabs, Shirley, N.Y.) at different concentration of 0, 0.1, 1, 3, 10, or 30 μg/mL at 37° C. for 30 min. Human IgG1 at corresponding concentrations was used as isotype control. The treated cells were washed twice with assay medium and four-hour cytotoxicity assay was performed as described in section 5.3.

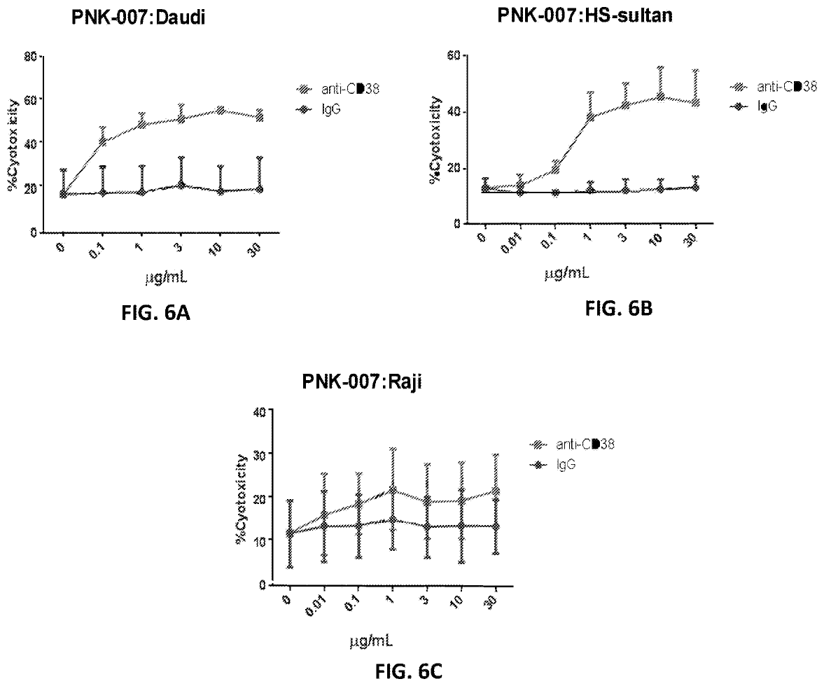

FIGS. 6A-6C show that the cytotoxicity of PNK cells against Daudi (FIG. 6A) and HS-Sultan cells (FIG. 6B) significantly increased in presence of daratumumab, compared with that in presence of IgG1 control, at E:T ratio of 1:1. But the cytotoxicity of PNK cells against Raji cells did not change significantly in presence of daratumumab, compared with that in presence of IgG1 control, at E:T ratio of 1:1. (FIG. 6C).

### 6.6. Example 6: Cytokine Secretion of PNK in Combination with Anti-CD38 Antibody Daratumumab

1×10⁵ PNK cells from three donors were incubated with HS-sultan cells, which were pre-treated with 10 μg/mL daratumumab or human IgG1, in 96-well U-bottom tissue culture plates at an E:T ratio of 1:1 in 200 μL of assay medium (see section 5.1). After 24 hour incubation at 37° C. and 5% CO₂, the supernatant was collected. Cytokine concentrations were determined by Luminex analysis using MILLIPLEX MAP magnetic bead kits (EMD Millipore, Billerica, Mass., Cat # HCD8MAG-15K-07 for GM-CSF, perforin, TNF-α, IL-10, granzyme A, granzyme B and IFN-γ; Cat # HCYTOMAG-60K-02 for MCP-1 and IFN-α2) according to the protocols provided by the manufacturer. Data were analyzed using Milliplex Xponent and Analyst software (EMD Millipore).

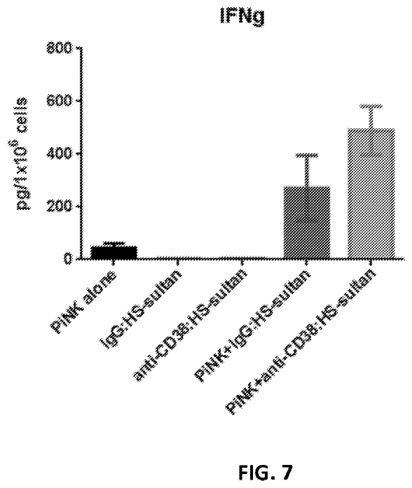

FIG. 7 shows increased IFN-γ secretion of PNK cells in presence of daratumumab-pretreated HS-sultan cells, compared with that of PNK cells alone or PNK cells in presence of IgG1-pretreated HS-sultan cells. Secretion of the other cytokines measured did not change significantly (data not shown).

### 6.7. Example 7: Anti-CD38 Antibody Daratumumab Exhibited No Effect on Viability of PNK Cells In Vitro

PNK cells from three donors were treated with daratumumab at different concentration of 0, 0.01, 0.1, 1, 3, 10, 30, or 100 μg/mL at 37° C. for 30 min. Human IgG at corresponding concentrations was used as controls. After two times washing, the PNK cells were resuspended with assay medium (see section 5.1) at 1×10⁶ cells/mL then incubated at 37° C., 5% C₀₂ for 4 hours. Five microliter 1 mM TO-PRO-3 was added to the culture, followed by data collection by FACSCanto II.

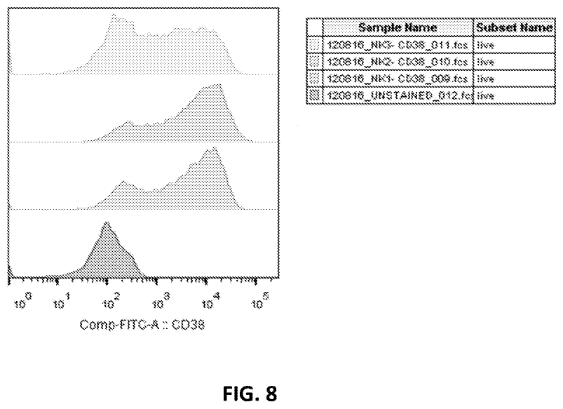

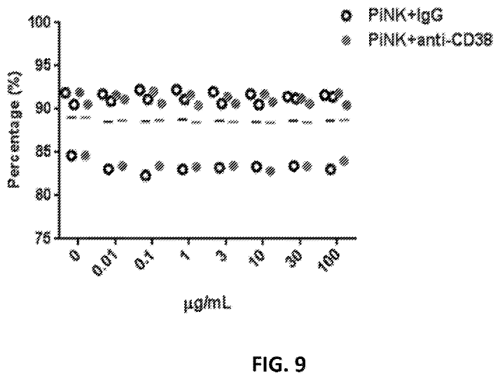

The percentage of CD38 expression on the PNK cells from the three donors was 75.2, 81.1, and 55.8%, respectively (FIG. 8). Daratumumab had no significant effect on viability of the PNK cells (n=3) in comparison with human IgG control (FIG. 9).

### 6.8. Example 8: Expression of CD20 on Human Lymphoma Daudi Cell Line

1×10⁵ Daudi cells were stained with PerCP-Cy5.5 mouse anti-human CD20 (BD, Cat #560736) at 4° C. for 15 min. Unstained tumor cells and tumor cells stained with PerCP mouse IgG2b, K isotype control (BD Bioscience, Cat #558304) were used as controls.

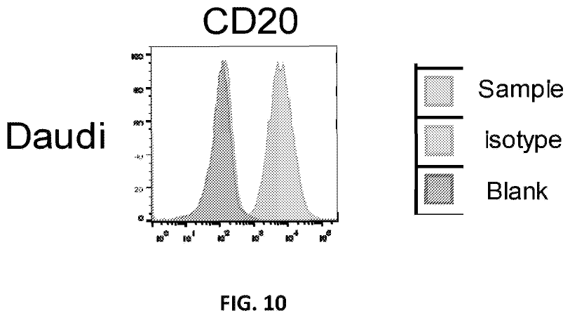

FIG. 10 depicts high percentage of CD20 expression on the Daudi cells.

### 6.9. Example 9: In Vitro Anti-Tumor Activity of PNK Cells in Combination with Anti-CD20 Antibody Rituxin® (Rituximab)

1×10⁷ Daudi cells were first labeled with 7.5 μM PKH26 fluorescent dye, then treated with Rituxin® (rituximab, from Genentech, South San Francisco, Calif.) at different concentration of 0, 1, 10, 20, 30, or 100 μg/mL at 37° C. for 30 min. Human IgG1 at corresponding concentrations was used as isotype control. The treated cells were washed twice with assay medium and four-hour cytotoxicity assay was performed as described in section 5.3.

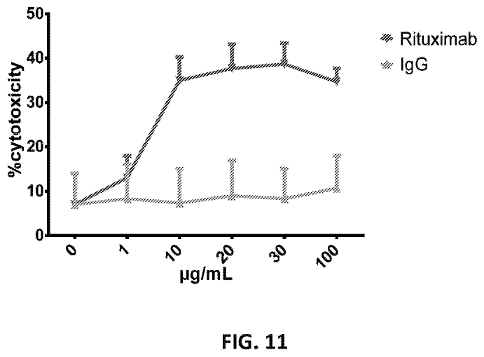

FIG. 11 shows that the cytotoxicity of PNK cells against Daudi cells significantly increased in presence of Rituxin®, compared with that in presence of IgG1 control, at E:T ratio of 1:1. The cytotoxicity of PNK cells was Rituxin® concentration-dependent.

### 6.10. Example 10: In Vivo Orthotopic Glioblastoma Tumor Model for Testing Combination Therapy

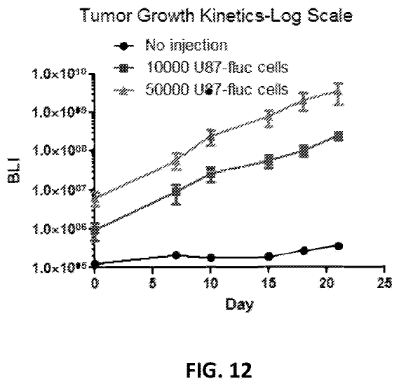

An orthotopic model for glioblastoma was generated using immuno-deficient NSG mice. Bioware® Brite Cell Line U87 MG-Red-FLuc (Cat # BW124577) was purchased from Perkin Elmer (Waltham, Mass.). Ten thousand (group 1, n=3) or fifty thousand (group 2, n=3) U87 MG-Red-FLuc cells were intracranially injected into immuno-deficient NSG mice at Day 0. A naive NSG mouse without injection of U87 MG-Red-FLuc cells was used as a control. Bioluminescence signal was monitored on Day 7, 10, 15, 18, and 21. NSG mice were anesthetized using Isoflurane. Ten minutes before imaging, luciferin was intraperitoneally injected into the animals. Live bioluminescence signal was determined by IVIS Spectrum In Vivo Imaging System (Perkin Elmer, Cat #124262). FIG. 12 shows tumor growth kinetics in the NSG mice that were injected with 10,000 or 50,000 U87 MG-Red-FLuc cells, compared with the control naive NSG mouse.

PNK cells and Unituxin® are administered to the orthotopic glioblastoma model in three different dosing regimens. For sequential administration, on Day 0, 10,000 U87 MG-Red-FLuc cells are injected intracranially; on Day 4, 0-5 mg/kg Unituxin® is injected intracranially; on Day 5, 1×10⁶ PNK cells are injected intracranially or 10×10⁶ PNK cells are injected intravenously. For co-administration, on Day 0, 10,000 U87 MG-Red-FLuc cells are injected intracranially; on Day 5, 0-5 mg/kg Unituxin® is injected intracranially, and 1×10⁶ PNK cells are injected intracranially or 10×10⁶ PNK cells are injected intravenously. For pre-treatment, on Day −1, U87 MG-Red-FLuc cells are co-cultured with Unituxin®; on Day 0, 10,000 U87 MG-Red-FLuc cells with or without pretreatment with Unituxin® are injected intracranially; on Day 5, 1×10⁶ PNK cells are injected intracranially or 10×10⁶ PNK cells are injected intravenously. Survival, clinical symptom, body weight, and bioluminescence signal are monitored on Day 7, 10, 15, 18, 21, and 28.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed is:

1. A method of suppressing the proliferation of tumor cells comprising contacting the tumor cells with human placental perfusate cells and an antibody, wherein said placental perfusate cells comprise CD56⁺ natural killer (NK) cells.

2. The method of claim 1, wherein the tumor cells are blood cancer cells.

3. The method of claim 1, wherein the tumor cells are solid tumor cells.

4. The method of claim 1, wherein the tumor cells are glioblastoma cells, neuroblastoma cells, osteosarcoma cells, melanoma cells, ovarian cancer cells, primary ductal carcinoma cells, leukemia cells, acute T cell leukemia cells, acute myelogenous leukemia (AML) cells, chronic myelogenous leukemia (CML) cells, acute lymphocytic leukemia (ALL) cells, chronic lymphocytic leukemia (CLL) cells, non-hodgkin's lymphoma (NHL) cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

5. The method of claim 1, wherein said contacting is contacting in vitro.

6. The method of claim 1, wherein said contacting is contacting in vivo.

7. The method of claim 8, wherein said contacting is in a human.

8. The method of claim 1, wherein said placental perfusate cells are total nucleated cells from placental perfusate.

9. The method of claim 1, wherein said placental perfusate cells comprise at least about 50% CD56⁺ NK cells.

10. A method of suppressing the proliferation of tumor cells comprising contacting the tumor cells with human placental perfusate cells and an antibody, wherein said human placental perfusate cells comprise CD56⁺, CD16⁻ placenta-derived intermediate natural killer (PINK) cells.

11. The method of claim 10, wherein said contacting takes place in vitro.

12. The method of claim 10, wherein said contacting takes place in vivo.

13. The method of claim 12, wherein said contacting takes place in a human.

14. The method of claim 10, wherein said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

15. The method of claim 10, wherein said PINK cells are contacted with an immunomodulatory compound in an amount and for a time sufficient for said PINK cells to express detectably more granzyme B than an equivalent number of PINK cells not contacted with said immunomodulatory compound.

16. The method of claim 15, wherein said immunomodulatory compound is lenalidomide, pomalidomide, or thalidomide.

17. A method of suppressing the proliferation of tumor cells comprising contacting the tumor cells with combined natural killer (NK) cells and an antibody, wherein said combined NK cells comprise NK cells isolated from placental perfusate and NK cells isolated from umbilical cord blood, and wherein said umbilical cord blood is isolated from the placenta from which said placental perfusate is obtained.

18. The method of claim 17, wherein said contacting takes place in vitro.

19. The method of claim 17, wherein said contacting takes place in vivo.

20. The method of claim 19, wherein said contacting takes place in a human.

21. The method of claim 17, wherein said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

22. The method of claim 17, wherein said combined NK cells comprise:
a detectably higher number of CD3⁻CD56⁺CD16⁻ NK cells than an equivalent number of NK cells from peripheral blood;
a detectably lower number of CD3⁻CD56⁺CD16⁺ NK cells than an equivalent number of NK cells from peripheral blood;
a detectably higher number of CD3⁻CD56⁺KIR2DL2/L3⁺ NK cells than an equivalent number of NK cells from peripheral blood;
a detectably lower number of CD3⁻CD56⁺NKp46⁺ NK cells than an equivalent number of NK cells from peripheral blood;
a detectably higher number of CD3⁻CD56⁺NKp30⁺ NK cells than an equivalent number of NK cells from peripheral blood;
a detectably higher number of CD3⁻CD56⁺2B4⁺ NK cells than an equivalent number of NK cells from peripheral blood; or
a detectably higher number of CD3⁻CD56⁺CD94⁺ NK cells than an equivalent number of NK cells from peripheral blood.

23. The method of claim 22, wherein said NK cells have not been cultured.

24. The method of claim 17, wherein said combined NK cells comprise:
a detectably lower number of CD3⁻CD56⁺KIR2DL2/L3⁺ NK cells than an equivalent number of NK cells from peripheral blood;
a detectably higher number of CD3⁻CD56⁺NKp46⁺ NK cells than an equivalent number of NK cells from peripheral blood;
a detectably higher number of CD3⁻CD56⁺NKp44⁺ NK cells than an equivalent number of NK cells from peripheral blood;
a detectably higher number of CD3⁻CD56⁺NKp30⁺ NK cells than an equivalent number of NK cells from peripheral blood.

25. The method of claim 24, wherein said NK cells have been cultured.

26. The method of claim 25, wherein said NK cells have been cultured for about 21 days.

27. A method of treating an individual having tumor cells, comprising administering to said individual a therapeutically effective amount of human placental perfusate cells and an antibody, wherein said human placental perfusate cells comprise CD56⁺ NK cells.

28. The method of claim 27, wherein the tumor cells are blood cancer cells.

29. The method of claim 27, wherein the tumor cells are solid tumor cells.

30. The method of claim 27, wherein the tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

31. The method of claim 27, wherein said human placental perfusate cells and/or said antibody are administered intravenously.

32. The method of claim 27, wherein said human placental perfusate cells and/or said antibody are administered intracranially.

33. The method of claim 27, wherein said human placental perfusate cells and/or said antibody are administered intrathecally.

34. The method of claim 27, wherein said human placental perfusate cells are total nucleated cells from placental perfusate.

35. The method of claim 27, wherein said human placental perfusate cells comprise at least about 50% CD56⁺ NK cells.

36. A method of treating an individual comprising tumor cells comprising administering to said individual a therapeutically effective amount of human placental perfusate cells and an antibody, wherein said human placental perfusate cells comprise CD56⁺, CD16⁻ PINK cells.

37. The method of claim 36, wherein said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

38. The method of claim 36, wherein said PINK cells are contacted with an immunomodulatory compound in an amount and for a time sufficient for said PINK cells to express detectably more granzyme B than an equivalent number of PINK cells not contacted with said immunomodulatory compound.

39. The method of claim 38, wherein said immunomodulatory compound is lenalidomide, pomalidomide, or thalidomide.

40. The method of claim 36, wherein said human placental perfusate cells and/or said antibody are administered intravenously.

41. The method of claim 36, wherein said human placental perfusate cells and/or said antibody are administered intracranially.

42. The method of claim 36, wherein said human placental perfusate cells and/or said antibody are administered intrathecally.

43. A method of treating an individual having tumor cells, comprising administering to said individual a therapeutically effective amount of combined NK cells and an antibody, wherein said combined NK cells comprise PINK cells isolated from placental perfusate and NK cells isolated from umbilical cord blood, and wherein said umbilical cord blood is isolated from the placenta from which said placental perfusate is obtained.

44. The method of claim 43, wherein said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

45. The method of claim 43, wherein said combined NK cells comprise:
a detectably higher number of CD3−CD56+CD16− NK cells than an equivalent number of NK cells from peripheral blood;
a detectably lower number of CD3−CD56+CD16+NK cells than an equivalent number of NK cells from peripheral blood;
a detectably higher number of CD3−CD56+KIR2DL2/L3+NK cells than an equivalent number of NK cells from peripheral blood;
a detectably lower number of CD3−CD56+NKp46+NK cells than an equivalent number of NK cells from peripheral blood;
a detectably higher number of CD3−CD56+NKp30+NK cells than an equivalent number of NK cells from peripheral blood;
a detectably higher number of CD3−CD56+2B4+NK cells than an equivalent number of NK cells from peripheral blood; or
a detectably higher number of CD3−CD56+CD94+NK cells than an equivalent number of NK cells from peripheral blood.

46. The method of claim 45, wherein said NK cells have not been cultured.

47. The method of claim 43, wherein said combined NK cells comprise:
a detectably lower number of CD3⁻CD56⁺KIR2DL2/L3⁺ NK cells than an equivalent number of NK cells from peripheral blood;
a detectably higher number of CD3⁻CD56⁺NKp46⁺ NK cells than an equivalent number of NK cells from peripheral blood;
a detectably higher number of CD3⁻CD56⁺NKp44⁺ NK cells than an equivalent number of NK cells from peripheral blood;
a detectably higher number of CD3⁻CD56⁺NKp30⁺ NK cells than an equivalent number of NK cells from peripheral blood.

48. The method of claim 47, wherein said NK cells have been cultured.

49. The method of claim 48, wherein said NK cells have been cultured for about 21 days.

50. The method of claim 43, wherein said combined NK cells and/or said antibody are administered intravenously.

51. The method of claim 43, wherein said combined NK cells and/or said antibody are administered intracranially.

52. The method of claim 43, wherein said combined NK cells and/or said antibody are administered intrathecally.

53. The method of claim 27, wherein said human placental perfusate cells comprise at least about 50% CD56⁺ PINK cells.

54. The method of claim 36, wherein said human placental perfusate cells comprise at least about 50% CD56⁺, CD16⁻ PINK cells.

55. The method of claim 27, wherein said PINK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, or hsa-miR-99a at a detectably higher level than peripheral blood NK cells.

56. The method of claim 36, wherein said PINK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, or hsa-miR-99a at a detectably higher level than peripheral blood NK cells.

57. The method of claim 43, wherein said PINK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, or hsa-miR-99a at a detectably higher level than peripheral blood NK cells.

58. The method of claim 27, wherein said PINK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

59. The method of claim 36, wherein said PINK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

60. The method of claim 43, wherein said PINK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

61. A method of treating an individual comprising tumor cells comprising administering to said individual a therapeutically effective amount of CD56⁺, CD16⁻ PINK cells and an antibody.

62. The method of claim 61, wherein the CD56⁺, CD16⁻ PINK cells are generated from CD34⁺ hematopoietic stem cells by a two-step expansion and differentiation method.

63. The method of claim 61, wherein said tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells.

64. The method of claim 61, wherein said PINK cells are contacted with an immunomodulatory compound in an amount and for a time sufficient for said PINK cells to express detectably more granzyme B than an equivalent number of PINK cells not contacted with said immunomodulatory compound.

65. The method of claim 64, wherein said immunomodulatory compound is lenalidomide, pomalidomide, or thalidomide.

66. The method of claim 61, wherein said PINK cells and/or said antibody are administered intravenously.

67. The method of claim 61, wherein said PINK cells and/or said antibody are administered intracranially.

68. The method of claim 61, wherein said PINK cells and/or said antibody are administered intrathecally.

69. The method of any one of claims 1, 10, 17, 27, 36, 43, or 61, wherein said antibody is selected from the group consisting of an anti-disialoganglioside (anti-GD2) antibody, an anti-CD38 antibody, an anti-SLAMF7 antibody, an anti-CD 20 antibody, an anti-HER2 antibody, an anti-PD-L1 antibody, and an anti-EGFR antibody.

70. The method of claim 69, wherein the antibody is an anti-GD2 antibody.

71. The method of claim 70, wherein the anti-GD2 antibody is dinutuximab.

72. The method of claim 69, wherein the antibody is an anti-CD38 antibody.

73. The method of claim 72, wherein the anti-CD38 antibody is daratumumab.

74. The method of claim 69, wherein the antibody is an anti-SLAMF7 antibody.

75. The method of claim 74, wherein the anti-SLAMF7 antibody is elotuzumab.

76. The method of claim 69, wherein the antibody is an anti-CD 20 antibody.

77. The method of claim 76, wherein the anti-CD 20 antibody is obinutuzumab, ofatumumab, or rituximab.

78. The method of claim 69, wherein the antibody is an anti-HER2 antibody.

79. The method of claim 78, wherein the anti-HER2 antibody is trastuzumab.

80. The method of claim 69, wherein the antibody is an anti-PD-L1 antibody.

81. The method of claim 80, wherein the anti-PD-L1 antibody is atezolizumab or avelumab.

82. The method of claim 69, wherein the antibody is an anti-EGFR antibody.

83. The method of claim 82, wherein the anti-EGFR antibody is cetuximab.

84. The method of claim 27 or 36, wherein the human placenta perfusate cells and the antibody are administered concurrently.

85. The method of claim 27 or 36, wherein the human placenta perfusate cells and the antibody are administered sequentially.

86. The method of claim 43, wherein the combined NK cells and the antibody are administered concurrently.

87. The method of claim 43, wherein the combined NK cells and the antibody are administered sequentially.

88. The method of claim 61, wherein the PINK cells and the antibody are administered concurrently.

89. The method of claim 61, wherein the PINK cells and the antibody are administered sequentially.
